SEQUENCE LISTING (33 sequences)

SEQ ID NO: 1 - protein, 622 aa, Homo sapiens
  MISC_FEATURE (1)..(622): Prolactin receptor (PrlR)
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 2 - protein, 638 aa, Homo sapiens
  MISC_FEATURE (1)..(638): Growth Hormone Receptor (GHR)
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 3 - protein, 198 aa, Homo sapiens
  MISC_FEATURE (1)..(198): Suppressor Of Cytokine Signalling 2 (SOCS2)
MTLRCLEPSGNGGEGTRSQWGTAGSAEEPSPQAARLAKALRELGQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPSLQHLCRLTINKCTGAIWGLPLPTRLKDYLEEYKFQV

SEQ ID NO: 4 - protein, 1807 aa, Homo sapiens
  MISC_FEATURE (1)..(1807): Tuberin / Tuberous sclerosis 2 protein (TCS2)
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 5 - protein, 227 aa, Homo sapiens
  MISC_FEATURE (1)..(227): wtPrl including signal peptide
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 6 - protein, 199 aa, Homo sapiens
  MISC_FEATURE (1)..(199): Mature wt-Prl
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 7 - protein, 199 aa, Homo sapiens
  MISC_FEATURE (1)..(199): Mature Prl (mutated S33A , Q73L, G129R, K190R)
LPICPGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 8 - protein, 199 aa, Homo sapiens
  MISC_FEATURE (1)..(199): Mature Prl (mutated S61A, D68N, Q73L, G129R, K190R)
LPICPGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 9 - protein, 191 aa, Homo sapiens [sequence omitted]

SEQ ID NO: 10 - protein, 191 aa, Homo sapiens [sequence omitted]

SEQ ID NO: 11 - protein, 7 aa, Artificial Sequence
  Linker
SGGSGGS

SEQ ID NO: 12 - protein, 15 aa, Artificial Sequence
  Linker
GGSGGSGGSGGSGGG

SEQ ID NO: 13 - protein, 18 aa, Artificial Sequence
  Linker
GGSGGSGGSGGSGGSGGS

SEQ ID NO: 14 - protein, 15 aa, Artificial Sequence
  Linker
GGGGSGGGGSGGGGS

SEQ ID NO: 15 - protein, 8 aa, Artificial Sequence
  Linker
EFAGAAAV

SEQ ID NO: 16 - protein, 198 aa, Homo sapiens
  MISC_FEATURE (1)..(198): N-terminally truncated Prl (mutated S33A, Q73L, G129R, K190R)
PICPGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 17 - protein, 197 aa, Homo sapiens
  MISC_FEATURE (1)..(197): N-terminally truncated Prl (mutated S33A, Q73L, G129R, K190R)
ICPGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 18 - protein, 196 aa, Homo sapiens
  MISC_FEATURE (1)..(196): N-terminally truncated Prl (mutated S33A, Q73L, G129R, K190R)
CPGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 19 - protein, 195 aa, Homo sapiens
  MISC_FEATURE (1)..(195): N-terminally truncated Prl (mutated S33A, Q73L, G129R, K190R)
PGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 20 - protein, 194 aa, Homo sapiens
  MISC_FEATURE (1)..(194): N-terminally truncated Prl (mutated S33A, Q73L, G129R, K190R)
GGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 21 - protein, 193 aa, Homo sapiens
  MISC_FEATURE (1)..(193): N-terminally truncated Prl (mutated S33A, Q73L, G129R, K190R)
GAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 22 - protein, 192 aa, Homo sapiens
  MISC_FEATURE (1)..(192): N-terminally truncated Prl (mutated S33A, Q73L, G129R, K190R)
AARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 23 - protein, 191 aa, Homo sapiens
  MISC_FEATURE (1)..(191): N-terminally truncated Prl (mutated S33A, Q73L, G129R, K190R)
ARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 24 - protein, 190 aa, Homo sapiens
  MISC_FEATURE (1)..(190): N-terminally truncated Prl (mutated S33A, Q73L, G129R, K190R)
RCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 25 - protein, 198 aa, Homo sapiens
  MISC_FEATURE (1)..(198): N-terminally truncated Prl (mutated S61A, D68N, Q73L, G129R, K190R)
PICPGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 26 - protein, 197 aa, Homo sapiens
  MISC_FEATURE (1)..(197): N-terminally truncated Prl (mutated S61A, D68N, Q73L, G129R, K190R)
ICPGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 27 - protein, 196 aa, Homo sapiens
  MISC_FEATURE (1)..(196): N-terminally truncated Prl (mutated S61A, D68N, Q73L, G129R, K190R)
CPGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 28 - protein, 195 aa, Homo sapiens
  MISC_FEATURE (1)..(195): N-terminally truncated Prl (mutated S61A, D68N, Q73L, G129R, K190R)
PGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 29 - protein, 194 aa, Homo sapiens
  MISC_FEATURE (1)..(194): N-terminally truncated Prl (mutated S61A, D68N, Q73L, G129R, K190R)
GGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 30 - protein, 193 aa, Homo sapiens
  MISC_FEATURE (1)..(193): N-terminally truncated Prl (mutated S61A, D68N, Q73L, G129R, K190R)
GAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 31 - protein, 192 aa, Homo sapiens
  MISC_FEATURE (1)..(192): N-terminally truncated Prl (mutated S61A, D68N, Q73L, G129R, K190R)
AARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 32 - protein, 191 aa, Homo sapiens
  MISC_FEATURE (1)..(191): N-terminally truncated Prl (mutated S61A, D68N, Q73L, G129R, K190R)
ARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 33 - protein, 190 aa, Homo sapiens
  MISC_FEATURE (1)..(190): N-terminally truncated Prl (mutated S61A, D68N, Q73L, G129R, K190R)
RCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

(19) United States
(12) Patent Application Publication
(10) Pub. No.: US 2015/0133383 A1
(43) Pub. Date: May 14, 2015

(54) METHOD FOR DIAGNOSIS AND TREATMENT OF PROLACTIN ASSOCIATED DISORDERS

(71) Applicant: ProRec Bio AB, Bromma (SE)

(72) Inventor: Gunnar Norstedt, Bromma (SE)

(21) Appl. No.: 14/400,430

(22) PCT Filed: May 13, 2013
(86) PCT No.: PCT/EP2013/059806

§ 371 Date: Nov. 11, 2014

Related U.S. Application Data
(60) Provisional application No. 61/646,086, filed on May 11, 2012.

(51) Int. Cl. G01N 33/574

(52) U.S. Cl. CPC G01N 33/57492; G01N 2333/72
USPC 514/11.3; 435/7.23; 514/11.5

(57) ABSTRACT

The present invention concerns methods and tools for determining a specific treatment of a prolactin associated disorder. The treatment is selected based on the expression pattern of growth hormonereceptor (GHR), prolactin receptor (PrlR) and the suppressors SOCS2 and TCS2.

### FIELD OF INVENTION

The present invention relate to the field of diagnostic and therapeutic methods in relation to proliferative disorders, in particular of disorders associated with imbalance in growth hormone and/or prolactin levels.

### BACKGROUND OF INVENTION

It has been known for many years that certain tumours depend on hormones for their growth [11]. This is e.g. the case with breast and prostate cancer where current treatment includes antagonists of estrogen and androgen receptors [1, 2] respectively. It is important to know prior to treatment if a tumour will respond to treatment and therefore most breast cancers are subjected to analysis of estrogen receptors to determine if they are estrogen responsive. In general, it is an emerging trend to measure certain markers in tumour samples to ensure that tumours will respond to treatment before treatment is commenced [3].

A number of technologies have been used to find so called tumour markers to find diagnostic and/or sub-classifying components in tumours [4, 5]. It is important to distinguish between diagnostic markers and markers useful to guide treatments. Techniques measuring a majority of expressed genes report differences in hundreds of expressed genes between normal tissues or various cancer types. From a practical point of view such extensive lists of components have limited value because of interpretation problems and generation data that requires advanced methods not available in the clinic. Furthermore, tumour specific mRNAs may not be relevant to guide treatment due to post-transcriptional modification prior to translation. For this reason, it is normally preferred to analyse translated proteins rather than mRNA if the object is to use markers to guide therapeutic treatment. Also in the case of protein analysis it is possible to find long lists of proteins that are expressed in a manner specific for a tumour. The challenge in this case is subsequently to select a minimal set of markers that will adequately correlate with or report if a tissue will respond or not to a treatment, in a method that can be adapted to techniques available for conventional clinical analytical procedures.

The hormone prolactin (Prl), which is a cytokine with several known biological functions, has been studied for several decades [6, 7]. For example, it is known to be a potent growth factor for mammary epithelium and has been associated with the development and growth of breast tumours and prostate tumours [8]. However, the inhibition of pituitary secretion of the hormone, e.g. by dopamine receptor agonists, has no effect on breast tumours and reduced production is insufficient for treatment of the disease. Therefore, it has been hypothesized [9] that blocking one or more of the actions of Prl by using Prl receptor antagonists would have beneficial medical effects. However, it has been difficult to substantiate the hypothesis that antagonizing Prl receptors really has a value in medical therapy. Several factors contribute to this uncertainty to predict medical utilities of Prl receptor antagonists:

First, the potencies of the Prl receptor antagonists that have been tested have been very different, making it difficult to draw reliable conclusion on the effect in various situations, where Prl and its receptor is involved. A great number of Prl mutants with varying numbers of mutations and combinations of these, have been disclosed, e.g. WO2009/003732 and WO2008/040758, but no specific Prl receptor antagonists have so far been identified which fulfill the criteria for medical use with sufficient effect.

Second, the Prl receptor antagonists identified to date have been mutated Prl variants, binding of such compounds are suspected to display species specificity, because Prl receptors have amino acid sequence variations in the ligand receptor surface. Accordingly, the large species differences in Prl actions present problems, because blocking the receptor in one species may cause biological effects only in that species and this means that e.g. in vivo animal data may have only limited value, or even no relevance at all, for the human case, unless produced in systems carefully selected for relevant conclusions to be drawn.

Third, growth hormone (GH) binds to Prl receptors meaning that any Prl receptor antagonist in principal can block GH actions. Naturally, this further complicates predictions on the use of putative Prl antagonists.

For these reasons, the situation with regard to future medical utilities of Prl receptor antagonists is considered unclear by those of skill in the art. As mentioned above, attempts to translate to medical use from experimental systems (e.g. mice and rats) are complicated by the existence of species differences. Prl and Prl receptor blockers may act on cultured cells, but the relation of cultured cells to cells in an intact organism or organ is continuously debated. Therefore, even if a substance in literature is described as a high affinity compound, its function in humans cannot be foreseen on a level where more definite conclusions can be drawn. From the above said, there are reasons to be concerned with the type of broad claims on the use of Prl receptor antagonists that can be found in the literature, both with regard to the range of medical indications as well as the huge number of substances covered by generic formula.

Prl binding to its membrane bound receptor activates cell signals to create a biological response. The key step to activate a signal is the Prl induced receptor dimerization, i.e. two receptors are brought in proximity which activates intra-cellular signaling systems. Since dimerization is a critical step, previous work has shown that mutations in the 199 amino acid long sequence of human Prl can prevent receptor dimerization. Replacement of glycine in position 129 to arginine (Prl G129R) is an example of such a mutation creating a Prl receptor antagonist, as this mutation prevents receptor dimerization. However, [10] it has been shown that the G129R mutation in Prl changes binding characteristics of the Prl molecules so that this compound has relatively low affinity to the Prl receptor. As described e.g. in the publications mentioned above (WO2009/003732 and WO2008/040758), as well as Liu et al[10], various Prl receptor antagonists can be developed by changing a number of amino acids in Prl. As these mutations can be selected and combined in a very large number of manners, the reader is left with insufficient teaching for designing a therapy based on this concept, both with regard to the selection of efficient compounds as well as specific medical conditions where therapy can be expected to be of particular value. Accordingly there is a need for improved methods for guiding therapy.

### SUMMARY OF INVENTION

The present invention, in one aspect concerns a composition comprising at least one prolactin receptor antagonist and/or at least one growth hormone receptor antagonist, for use in a method of treatment of a prolactin-associated disorder in an individual, the method comprising the steps of:

a) providing a sample of tumour tissue obtained from the individual,
b) determining in said sample, the expression level of the polypeptides growth hormone receptor (GHR), prolactin receptor (PrlR), suppressor of cytokine signalling 2 (SOCS2) and tuberous sclerosis complex 2 (TSC2),
c) comparing the expression levels of step b) with the expression level of a control tissue,
d) assessing a treatment regime by correlating the results of step c) with the corresponding expression pattern of table 1,
e) administering to the individual a therapeutically effective amount of said composition as determined in step d).

In one aspect the invention concerns a method of treatment of a prolactin-associated disorder in an individual in need thereof, the method comprising the steps of:

a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) assessing a treatment regime, administering to the individual a therapeutically effective amount of a prolactin receptor antagonist and/or a growth hormone receptor antagonist.

In one aspect the invention concerns a method for selecting treatment of a prolactin-associated disorder in an individual, the method comprising the steps of:

a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) selecting a treatment regime based on table 1.

In one aspect the invention concerns a method for diagnosing a prolactin-associated disorder in an individual, the method comprising the steps of:

a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue, wherein an expression pattern equal to the expression patterns 3, 4, 5, 6, 9, 12, 13, 14, 15, 18, 21, 22, 23, 24, 27, 30, 31, 32, 33, 36, 39, 41, 42, 45, 57, 58, 59, 60, 63, 66, 67, 68, 69, 72, 76, 77, 78 or 81 of table 1 is indicative of a prolactin associated disorder.

In one aspect the invention concerns a computer implemented method for selecting treatment of a prolactin-associated disorder, the method comprising the steps of:

a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) selecting a treatment regime based on table 1.

In one aspect the invention concerns a computer implemented method for diagnosing a prolactin-associated disorder, the method comprising the steps of:

a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue, wherein an expression pattern equal to the expression patterns 3, 4, 5, 6, 9, 12, 13, 14, 15, 18, 21, 22, 23, 24, 27, 30, 31, 32, 33, 36, 39, 41, 42, 45, 57, 58, 59, 60, 63, 66, 67, 68, 69, 72, 76, 77, 78 or 81 of table 1 is indicative of a prolactin associated disorder.

In one aspect the invention concerns a computer program product having a computer readable medium, said computer program product suitable for selecting a treatment of a prolactin associated disorder in a subject based on expression patterns of the polypeptides growth hormone receptor (GHR), prolactin receptor (PrlR), suppressor of cytokine signalling 2 (SOCS2) and tuberous sclerosis complex 2 (TSC2) in tumour tissue in a sample obtained from said subject, said computer program product comprising means for carrying out all the steps of the method as defined herein below.

In one aspect the invention concerns the use of a composition comprising at least one prolactin receptor antagonist and/or at least one growth hormone receptor antagonist, for the preparation of a medicament for the treatment of a prolactin-associated disorder in an individual, said use comprising the steps of:

a) providing a sample of tumour tissue obtained from the individual,
b) determining in said sample, the expression level of the polypeptides growth hormone receptor (GHR), prolactin receptor (PrlR), suppressor of cytokine signalling 2 (SOCS2) and tuberous sclerosis complex 2 (TSC2),
c) comparing the expression levels of step b) with the expression level of a control tissue,
d) assessing a treatment regime by correlating the results of step c) with the corresponding expression pattern of table 1,
e) administering to the individual a therapeutically effective amount of said composition as determined in step d).

In n one aspect the invention concerns a kit comprising the composition as defined herein, and instructions for use.

### DESCRIPTION OF DRAWINGS

FIG. 1: Reduction of TSC2 increases prolactin receptor levels.

The myosarcoma cell line CRL-2620 (TSC2−/+) was obtained from ATCC and were cultured in DMEM with 10% serum. Cells were electroporated (AMAXA instrument, setting E014) with siRNA for TSC2 and with control siRNA. Approximately 600 000 cells were distributed into 2 cm culture dishes. After one day, cells were serum starved over night, followed by a wash procedure and subsequent preparation of protein lysates. The protein extracts were separated on a 12% SDS-PAGE gels, blotted onto PVF membranes and probed with primary antibodies. The membranes were further probed with HRP conjugated secondary antibodies. The antibodies used were Anti-prolactin receptor M170 from Santa Cruz and a GH receptor antibody B10 also from Santa Cruz. The Figure show triplicate determination of the GH receptor, prolactin receptor, SOCS3 and GAPDH. As demonstrated only the prolactin receptor was increased by TSC2 reduction. Densitometric scanning of the Western blot showed that the increase of prolactin receptor levels was approximately 6-fold.

FIG. 2: Increased levels of prolactin receptors in SOCS2 deficient mice.

Mice deficient of SOCS2 were used to analyse prolactin receptor (Prl) levels. Liver samples from control mice and from SOCS2 knock out mice were fixed and sectioned for immuno-histochemistry. The tissue sections were incubated with anti-prolactin receptor antibodies and detection of a secondary fluorescent antibody signal was made using fluorescence confocal microscopy. The image on the left shows staining in a control liver (WT) and to the right staining in a SOCS2 knock out (KO) liver.

FIG. 3: The prolactin receptor antagonist (SEQ ID NO: 24) blocks the phosphorylation of STAT5. MCF7 breast cancer cells were cultured in DMEM medium with 10% fetal bovine serum (FBS) and semi-confluent cells were starved in DMEM without FBS for 18 h before hormone stimulation. The cells were stimulated for 10 minutes with 20 nM prolactin combined with increasing amounts of the prolactin receptor antagonist (SEQ ID NO: 24). Phosphorylated STAT5 was measured in protein extract separated on SDS-PAGE gels using the Western blot technique.

FIG. 4: The prolactin receptor antagonist (SEQ ID NO: 24) reduces cell proliferation in breast cancer cells. MCF7 breast cancer cells in DMEM medium with 10% fetal bovine serum (FBS) were seeded in 96 well plates. After 24 h the medium was changed to different volumes (50 ul and 250 ul) of starvation medium (DMEM without FBS). After 48 h, the cell were fixed with 2% formaldehyde, washed and stained with 0.04% crystal violet assay. Crystal violet was subsequently solubilized with 1% SDS and the absorbance was measured at 600 nM. Error bars represent SEM of 8 replicates. The cell numbers in high versus low volume (250 ul vs 50 ul) were analysed as well as the result of prolactin stimulation (20 nM) (panel A). Panel B displays the effect of different concentrations of the prolactin receptor antagonist (SEQ ID NO: 24) tested in cells kept in a low volume of culture medium. It was concluded that proliferation is larger in cells were the medium is of a low volume and that prolactin stimulates cell proliferation under conditions of diluted cell medium. The prolactin receptor antagonist (SEQ ID NO: 24) reduces proliferation in conditions when cell culture volume is small which may indicate a block of cell produced prolactin. Right hand column of panel B demonstrates cells cultured at high volume without ligand (i.e. no Prl and no PrlR-antagonist), demonstrating the volume of medium affects proliferation and that the antagonist works best when the volume is low, as Prl is otherwise diluted too much.

FIG. 5: Schematic overview of signalling and interactions in the GHR/PrlR/SOCS2/TCS2 system.

A) Normal situation/normal sensitivity; GH and Prl signals are controlled by the suppressors SOCS2 and TCS2

B) GH and/or Prl hyper sensitivity; loss of either SOCS2 or TSC2 increases signals from either or both GHR or PrlR

C) GH and/or Prl hypersensitivity; loss of SOCS2 or TSC2 leads to increased GH/Prl signals and increased GHR/PrlR

### DETAILED DESCRIPTION OF THE INVENTION

### Definitions

Adjuvant: Any substance whose admixture with an administered immunogenic determinant/antigen increases or otherwise modifies the immune response to said determinant.

Affinity: The interaction of most ligands with their binding sites can be characterized in terms of a binding affinity. In general, high affinity ligand binding results from greater intermolecular force between the ligand and its receptor while low affinity ligand binding involves less intermolecular force between the ligand and its receptor. In general, high affinity binding involves a longer residence time for the ligand at its receptor binding site than is the case for low affinity binding. High affinity binding of ligands to receptors is often physiologically important when some of the binding energy can be used to cause a conformational change in the receptor, resulting in altered behaviour of an associated ion channel or enzyme.

A ligand that can bind to a receptor, alter the function of the receptor and trigger a physiological response is called an agonist for that receptor. Agonist binding to a receptor can be characterized both in terms of how much physiological response can be triggered and the concentration of the agonist that is required to produce the physiological response. High affinity ligand binding implies that a relatively low concentration of a ligand is adequate to maximally occupy a ligand binding site and trigger a physiological response. Low affinity binding implies that a relatively high concentration of a ligand is required before the binding site is maximally occupied and the maximum physiological response to the ligand is achieved. Ligand binding is often characterized in terms of the concentration of ligand at which half of the receptor binding sites are occupied, known as the dissociation constant (k_{d}). Affinity is also the strength of binding between receptors and their ligands, for example between an antibody and its antigen.

Agonist: An agonist is a compound capable of increasing or effecting the activity of a receptor. Specifically, a Vps10p-domain receptor agonist is a compound capable of binding to one or more of binding sites of a Vps10p-domain receptor thereby inducing the same physiological response as a given endogenous agonist ligand compound.

Antagonist: An antagonist is in this case synonymous with an inhibitor. An antagonist is a compound capable of decreasing the activity of an effector such as a receptor. Specifically, a PrlR or GHR antagonist is a compound capable of binding to one or more of binding sites of PrlR or GHR respectively thereby inhibiting binding of another ligand thus inhibiting a physiological response.

Antibody: The term “antibody” as referred to herein includes whole antibodies and any antigen binding fragment (i.e., “antigen-binding portion”) or single chain thereof.

Polyclonal antibody: Polyclonal antibodies are a mixture of antibody molecules recognising a specific given antigen, hence polyclonal antibodies may recognise different epitopes within said antigen.

Aromatic group: the term “aromatic group” or “aryl group” means a mono- or polycyclic aromatic hydrocarbon group.

Bioreactive composition or biologically active or biological activity: The terms as used herein refers to effect of any compound or substance which may be used in connection with an application that is therapeutic or otherwise useful according to this invention.

Fc fragment: The term “an Fc fragment of a mammalian antibody” as used herein means a constant region, i.e. Fc fragment of a mammalian antibody or a fragment thereof wherein such mammalian antibody may be selected from IgM, IgG, IgA, IgD and IgE from a mammal, such as a primate, e.g. human, abe, or monkey; an equine, e.g. horse. A typical Fc fragment of a mammalian antibody is a recombinant Fc fragment of a human antibody, such as a recombinant Fc fragment of a human IgG antibody.

In the present context, the term “a variant of an Fc fragment of a mammalian antibody” or “Fc variant” (used interchangeably throughout the present description) as used herein means the Fc fragment of a mammalian antibody, wherein one or more amino acid residues, such as 1-10 amino acid residues, of the Fc fragment have been substituted by other amino acid residues and/or wherein one or more amino acid residues, such as 1-10 amino acid residues, have been deleted from the Fc fragment and/or wherein one or more amino acid residues, such as 1-10 amino acid residues, have been added to the Fc fragment and/or wherein one or more amino acid residues, such as 1-10 amino acid residues, in the Fc fragment have been modified. Such addition or deletion of amino acid residues can take e.g. place at the N-terminal of the Fc fragment and/or at the C-terminal of the Fc fragment. Native refers to an Fc that has not been modified by a human. WO 96/32478 describes exemplary Fc variants. Thus, the term “Fc variant” in one embodiment comprises a molecule or sequence that is humanized from a non-human native Fc. Furthermore, a native Fc comprises sites that may be removed because they provide structural features or biological activity that are not required for the fusion molecules of the present invention.

Fragments: The polypeptide fragments according to the present invention, including any functional equivalents thereof, may in one embodiment comprise less than 500 amino acid residues, such as less than 450 amino acid residues, for example less than 400 amino acid residues, such as less than 350 amino acid residues, for example less than 300 amino acid residues, for example less than 250 amino acid residues, such as less than 240 amino acid residues, for example less than 225 amino acid residues, such as less than 200 amino acid residues, for example less than 180 amino acid residues, such as less than 160 amino acid residues, for example less than 150 amino acid residues, such as less than 140 amino acid residues, for example less than 130 amino acid residues, such as less than 120 amino acid residues, for example less than 110 amino acid residues, such as less than 100 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues, such as less than 45 amino acid residues, for example less than 40 amino acid residues, such as 35 amino acid residues, for example 30 amino acid residues, such as 25 amino acid residues, such as 20 amino acid residues, for example 15 amino acid residues, such as 10 amino acid residues, for example 5 contiguous amino acid residues of an amino acid sequence selected from the group consisting of SEQ ID NOs: 1, 2, 3, 4, 5, 6, 7, 8, 9, 10, 16, 17, 18, 19, 20, 21, 22, 23, 24, 25, 26, 27, 28, 29, 30, 31, 32, 32 and 33 or a variant thereof being at least 70% (e.g. at least 85%, 90%, 95%, 97%, 98%, or 99%) identical to said sequences. Also, the polypeptide fragments according to the present invention, including any functional equivalents thereof, may in one embodiment comprise more than 5 amino acid residues, such as more than 10 amino acid residues, for example more than 15 amino acid residues, such as more than 20 amino acid residues, for example more than 25 amino acid residues, for example more than 50 amino acid residues, such as more than 75 amino acid residues, for example more than 100 amino acid residues, such as more than 125 amino acid residues, for example more than 150 amino acid residues, such as more than 175 amino acid residues of an amino acid sequence selected from the group consisting of SEQ ID NOs: 1, 2, 3, 4, 5, 6, 7, 8, 9, 10, 16, 17, 18, 19, 20, 21, 22, 23, 24, 25, 26, 27, 28, 29, 30, 31, 32, 32 and 33 or a variant thereof being at least 60% (e.g. at least 65%, 70%, 80%, 85%, 90%, 95%, 97%, 98%, or at least 99%) identical to said sequences.

Functional equivalency: “Functional equivalency” as used in the present invention is, according to one preferred embodiment, established by means of reference to the corresponding functionality of a predetermined fragment of the sequence.

Functional equivalents or variants of a polypeptide of the invention, or a fragment thereof will be understood to exhibit amino acid sequences gradually differing from the preferred predetermined polypeptide or fragment sequence respectively, as the number and scope of insertions, deletions and substitutions including conservative substitutions increase, while retaining the biological activity of a polypeptide in this context. This difference is measured as a reduction in identity between the preferred predetermined sequence and the fragment or functional equivalent.

A non-conservative substitution leading to the formation of a functionally equivalent fragment of a Prl polypeptide, or a fragment thereof would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Variants obtained by substitution of amino acids may in one preferred embodiment be made based upon the hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

Mutagenesis of a preferred predetermined Prl polypeptide, or a fragment thereof, can be conducted by making amino acid insertions, usually on the order of about from 1 to 10 amino acid residues, preferably from about 1 to 5 amino acid residues, or deletions of from about from 1 to 10 residues, such as from about 2 to 5 residues.

In one embodiment the ligand of binding site 1, 2 or 3 is an oligopeptide synthesised by automated synthesis. Any of the commercially available solid-phase techniques may be employed, such as the Merrifield solid phase synthesis method, in which amino acids are sequentially added to a growing amino acid chain (see Merrifield, J. Am. Chem. Soc. 85:2149-2146, 1963).

Equipment for automated synthesis of polypeptides is commercially available from suppliers such as Applied Biosystems, Inc. of Foster City, Calif., and may generally be operated according to the manufacturer's instructions. Solid phase synthesis will enable the incorporation of desirable amino acid substitutions into any fragment of Prl according to the present invention. It will be understood that substitutions, deletions, insertions or any subcombination thereof may be combined to arrive at a final sequence of a functional equivalent. Insertions shall be understood to include amino-terminal and/or carboxyl-terminal fusions, e.g. with a hydrophobic or immunogenic protein or a carrier such as any polypeptide or scaffold structure capable as serving as a carrier.

Oligomers including dimers including homodimers and heterodimers of fragments of PrlR antagonists and/or GHR antagonists according to the invention are also provided and fall under the scope of the invention. Prl polypeptides and fragments, functional equivalents and variants thereof can be produced as homodimers or heterodimers with other amino acid sequences or with native PrlR antagonist and/or GHR antagonist sequences. Heterodimers include dimers containing immunoreactive PrlR antagonist and/or GHR polypeptide antagonist fragments as well as fragments that need not have or exert any biological activity.

Prl polypeptides, or fragments and variants thereof may be synthesised both in vitro and in vivo. Methods for in vitro synthesis are well known, and methods being suitable or suitably adaptable to the synthesis in vivo of PrlR antagonists and/or GHR antagonists are also described in the prior art. When synthesized in vivo, a host cell is transformed with vectors containing DNA encoding a PrlR peptide based antagonist and/or GHR peptide antagonist or a fragment thereof. A vector is defined as a replicable nucleic acid construct. Vectors are used to mediate expression of Prl polypeptides, and/or fragments and variants. An expression vector is a replicable DNA construct in which a nucleic acid sequence encoding the predetermined PrlR and/or GHR antagonist fragment, or any functional equivalent thereof that can be expressed in vivo, is operably linked to suitable control sequences capable of effecting the expression of the fragment or equivalent in a suitable host. Such control sequences are well known in the art. Both prokaryotic and eukaryotic cells may be used for synthesising ligands.

Cultures of cells derived from multicellular organisms however represent preferred host cells. In principle, any higher eukaryotic cell culture is workable, whether from vertebrate or invertebrate culture. Examples of useful host cell lines are VERO and HeLa cells, Chinese hamster ovary (CHO) cell lines, and WI38, BHK, COS-7, 293 and MDCK cell lines. Preferred host cells are eukaryotic cells known to synthesize endogenous inhibitors. Cultures of such host cells may be isolated and used as a source of the fragment, or used in therapeutic methods of treatment, including therapeutic methods aimed at promoting or inhibiting a growth state, or diagnostic methods carried out on the human or animal body.

In vitro/in vivo: the terms are used in their normal meaning.

Ligand: a substance, compound or biomolecule such as a protein including receptors, that is able to bind to and form a complex with (a second) biomolecule to serve a biological purpose. In a narrower sense, it is a signal triggering molecule binding to a site on a target protein, by intermolecular forces such as ionic bonds, hydrogen bonds and Van der Waals forces. The docking (association) is usually reversible (dissociation). Actual irreversible covalent binding between a ligand and its target molecule is rare in biological systems. As opposed to the meaning in metallo-organic and inorganic chemistry, it is irrelevant, whether or not the ligand actually binds at a metal site, as it is the case in hemoglobin. Ligand binding to receptors may alter the chemical conformation, i.e. the three dimensional shape of the receptor protein. The conformational state of a receptor protein determines the functional state of a receptor. The tendency or strength of binding is called affinity. Ligands include substrates, inhibitors, activators, non-self receptors, co-receptors and neurotransmitters.

Linker: The term “linker” as used herein means a valence bond or multifunctional moiety, such as a bifunctional moiety that separates the Prl receptor antagonist polypeptide and the pharmaceutically acceptable molecule conjugated to the Prl receptor antagonist polypeptide and resulting in increased half-life such as increased plasma half-life.

Pharmaceutical composition: The terms “pharmaceutical composition” or “drug” or “medicament” refer to any therapeutic or prophylactic use of a composition according to the invention, which composition may be used in the treatment (including the prevention, diagnosis, alleviation, or cure) of a malady, affliction, condition, disease or injury in a patient. Therapeutically useful genetic determinants, peptides, polypeptides and polynucleotides may be included within the meaning of the term pharmaceutical or drug. As defined herein, a “therapeutic composition”, “pharmaceutical composition” or “drug” or “medicament” is a type of bioactive composition.

Pharmaceutical composition: or drug, medicament or composition refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “pharmaceutical composition” and “medicament” preferably encompass an active composition as such or an inactive drug and the active metabolite.

Polymer: The term “polymer” as used herein means a molecule formed by covalent linkage of two or more monomers, wherein none of the monomers is an amino acid residue, except where the polymer is human albumin or another abundant plasma protein. The term “polymer” may be used interchangeably with the term “polymer molecule”. The term is intended to cover carbohydrate molecules attached by in vitro glycosylation. Carbohydrate molecules attached by in vivo glycosylation, such as N- or O-glycosylation (as further described below) are referred to herein as “an oligosaccharide moiety”. Except where the number of polymer molecules is expressly indicated, every reference to “a polymer”, “a polymer molecule”, “the polymer” or “the polymer molecule” as used in the present invention shall be a reference to one or more polymer molecule(s). The polymer may be a water soluble or water insoluble polymer, such as a PEG moiety. The PEG moiety may have an average size selected from the range of 500 Da to 200.000 Da, such as from 500 Da to 100.000 Da, such as from 2000 Da to 50.000 Da. Such PEG molecules may be retrieved from i.a. Shearwater Inc.

Prolactin associated disorder: The term ‘prolactin-associated disorder or disease’ or ‘prolactin related disorder or disease’ as used herein should be understood as disorders resulting from a malfunction in the prolactin or prolactin/growth hormone signaling system, such as a disorder selected from the group consisting of colon cancer, liver cancer, breast cancer, endometrial cancer, ovary cancer, prostate cancer, parathyroid cancer, benign breast tumour, leiomyoma, renal angiomyolipoma, acromegaly, hyperprolactinemia, obesity resulting from endocrine malfunction, lymphangioleiomyomatosis, lupus erythematosus, benign prostate tumour and peripartum cardiomyopathy.

Prolactin Receptor Antagonist: A prolactin receptor antagonist abbreviated PrlR-A, is to be understood in the context of its conventional meaning as a compound capable of inhibiting signaling through the prolactin receptor.

Purified antibody: The term a “purified antibody” is an antibody at least 60 weight percent of which is free from the polypeptides and naturally-occurring organic molecules with which it is naturally associated. Preferably, the preparation comprises antibody in an amount of at least 75 weight percent, more preferably at least 90 weight percent, and most preferably at least 99 weight percent.

Sensitizing: The term sensitizing as used herein is understood as a left shift of a dose-response curve.

Sequence identity: The term “sequence identity” or “identical” as used herein refers to a relationship between the sequences of two or more proteins, as determined by comparing the sequences. The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTP programs of Altschul, et al. (1990) J. Mol. Biol. 215:403-410.

In order to characterize the identity, subject sequences are aligned so that the highest order homology (match) is obtained. Based on these general principles, the “percent identity” of two nucleic acid sequences may be determined using the BLASTN algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site (http://www.ncbi.nlm.nih.gov), and using the default settings suggested here (i.e. Reward for a match=1; Penalty for a mismatch=−2; Strand option=both strands; Open gap=5; Extension gap=2; Penalties gap x_dropoff=50; Expect=10; Word size=11; Filter on). The BLASTN algorithm determines the % sequence identity in a range of overlap between two aligned nucleotide sequences.

Another preferred, non-limiting example of a mathematical algorithm utilized for the comparison of sequences is the CLUSTAL W (1.7) alignment algorithm (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680.). CLUSTAL W can be used for multiple sequence alignment preferably using BLOSUM 62 as scoring matrix. When calculating sequence identities, CLUSTAL W includes any gaps made by the alignment in the length of the reference sequence.

A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence.

Treatment: The term “treatment” as used herein refers to a method involving therapy including surgery of a clinical condition in an individual including a human or animal body. The therapy may be ameliorating, curative or prophylactic, i.e. reducing mental and behavioural symptoms.

Variants: The term “variants” as used herein refers to amino acid sequence variants said variants preferably having at least 60% identity, for example at least 63% identity, such as at least 66% identity, for example at least 70% sequence identity, for example at least 72% sequence identity, for example at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with any of the predetermined sequences.

Up-regulation of expression: a process leading to increased expression of genes, preferably of endogenous genes.

### DESCRIPTION OF THE INVENTION

Determining Sensitivity

The present invention is in one aspect related to determining the degree of sensitivity of a tumour for growth hormone (GH) and prolactin (Prl), in order to tailor a treatment response of said tumour with substances capable of blocking the actions of GH and Prl, respectively. This comprises the method and use of the present invention for substances blocking the Prl receptor.

As mentioned herein above, the present invention concerns a combination of four biomarkers whose protein expression level in a tumour correlates with increased sensitivity of a tissue to the hormones growth hormone (GH) and prolactin (Prl). These markers have the utility to determine if a tumour will respond to compositions interfering with GH and/or Prl signalling through these receptors. A pre-requisite for any type of hormone response is expression of a receptor in the tissue where drug action is wanted.

According to the present invention it is therefore essential to measure protein levels of both the GH receptor and the Prl receptor. As demonstrated herein it is however not sufficient to measure the level of GH receptor and Prl receptors in a tumour sample because this will not sufficiently address the issue if GH/Prl sensitivity has been changed in a tumour. It is shown herein that if the analysis of GH receptor (GHR) and Prl receptor (PrlR) is supplemented with measurements of two additional components; SOCS2 and TSC2, these four markers in combination can be used to determine the GH/Prl sensitivity of tumours, and thus to indicate a disease state and assess a suitable treatment regime of the disease.

As mentioned herein above, the present inventor has found a correlation between the expression pattern of the polypeptides Growth Hormone Receptor (GHR), Prolactin Receptor (PrlR) Suppressor Of Cytokine Signalling (SOCS) and Tuberous Sclerosis Complex (TSC) and a group of diseases and disorders with the common feature of being associated with prolactin (Prl) imbalance, which enables diagnosis and/or prognosis of the disorder and proposes a treatment.

The present invention thus concerns a method of selecting treatment of a prolactin related disorder, the method comprising determining if the expression pattern of the polypeptides GHR, PrlR, SOCS2 and TCS2 in a diseased tissue such as tumour tissue, differs from the expression pattern exhibited in a sample obtained from a comparable non-tumour tissue.

The expression pattern which comprises four different biomarkers can vary in three different ways—either by being essentially unchanged, or by being up—or downregulated in relation to a control. Thus, the expression pattern comprises 81 different outcomes summarised in table 1 below.

TABLE 1
Overview of possible expression patterns

| | Expression pattern | | | | | Treatment |
|---|---|---|---|---|---|---|
| No. | GHR | PrIR | SOCS2 | TCS2 | Indication(s) | regime |
| 1 | ↑ | ↑ | ↑ | ↑ | | NT |
| 2 | ↑ | ↑ | ↑ | ← | | NT |
| 3 | ↑ | ↑ | ↑ | ↓ | C, D, J, N | GHR-A/PrIR-A |
| 4 | ↑ | ↑ | ↓ | ↑ | C, D, J, N | GHR-A/PrIR-A |
| 5 | ↑ | ↑ | ↓ | ← | C, D, J, N | GHR-A/PrIR-A |
| 6 | ↑ | ↑ | ↓ | ↓ | C, D, J, N | GHR-A/PrIR-A |
| 7 | ↑ | ↑ | ← | ↑ | | NT |
| 8 | ↑ | ↑ | ← | ← | | NT |
| 9 | ↑ | ↑ | ← | ↓ | C, D, J, N | GHR-A/PrIR-A |
| 10 | ↑ | ← | ↑ | ↑ | | NT |
| 11 | ↑ | ← | ↑ | ← | | NT |
| 12 | ↑ | ← | ↑ | ↓ | C, D, J, N | GHR-A |
| 13 | ↑ | ← | ↓ | ↑ | C, D, J, N | GHR-A |
| 14 | ↑ | ← | ↓ | ← | C, D, J, N | GHR-A |
| 15 | ↑ | ← | ↓ | ↓ | C, D, J, N | GHR-A |
| 16 | ↑ | ← | ← | ↑ | | NT |
| 17 | ↑ | ← | ← | ← | | NT |
| 18 | ↑ | ← | ← | ↓ | C, D, J, N | GHR-A |
| 19 | ↑ | ↓ | ↑ | ↑ | | NT |
| 20 | ↑ | ↓ | ↑ | ← | | NT |
| 21 | ↑ | ↓ | ↑ | ↓ | C, D, J, N | GHR-A |
| 22 | ↑ | ↓ | ↓ | ↑ | C, D, J, N | GHR-A |
| 23 | ↑ | ↓ | ↓ | ← | C, D, J, N | GHR-A |
| 24 | ↑ | ↓ | ↓ | ↓ | C, D, J, N | GHR-A |
| 25 | ↑ | ↓ | ← | ↑ | | NT |
| 26 | ↑ | ↓ | ← | ← | | NT |
| 27 | ↑ | ↓ | ← | ↓ | C, D, J, N | GHR-A |
| 28 | ↓ | ↑ | ↑ | ↑ | | NT |
| 29 | ↓ | ↑ | ↑ | ← | | NT |
| 30 | ↓ | ↑ | ↑ | ↓ | C, D, J, N | PrIR-A |
| 31 | ↓ | ↑ | ↓ | ↑ | C, D, J, N | PrIR-A |
| 32 | ↓ | ↑ | ↓ | ← | C, D, J, N | PrIR-A |
| 33 | ↓ | ↑ | ↓ | ↓ | C, D, J, N | PrIR-A |
| 34 | ↓ | ↑ | ← | ↑ | | NT |
| 35 | ↓ | ↑ | ← | ← | | NT |
| 36 | ↓ | ↑ | ← | ↓ | C, D, J, N | PrIR-A |
| 37 | ↓ | ← | ↑ | ↑ | | NT |
| 38 | ↓ | ← | ↑ | ← | | NT |
| 39 | ↓ | ← | ↑ | ↓ | C, D, J, N | PrIR-A |
| 40 | ↓ | ← | ↓ | ↑ | | NT |
| 41 | ↓ | ← | ↓ | ← | C, D, J, N | NT |
| 42 | ↓ | ← | ↓ | ↓ | C, D, J, N | PrIR-A |
| 43 | ↓ | ← | ← | ↑ | | NT |
| 44 | ↓ | ← | ← | ← | | NT |
| 45 | ↓ | ← | ← | ↓ | C, D, J, N | PrIR-A |
| 46 | ↓ | ↓ | ↑ | ↑ | | NT |
| 47 | ↓ | ↓ | ↑ | ← | | NT |
| 48 | ↓ | ↓ | ↑ | ↓ | | NT |
| 49 | ↓ | ↓ | ↓ | ↑ | | NT |
| 50 | ↓ | ↓ | ↓ | ← | | NT |
| 51 | ↓ | ↓ | ↓ | ↓ | | NT |
| 52 | ↓ | ↓ | ← | ↑ | | NT |
| 53 | ↓ | ↓ | ← | ← | | NT |
| 54 | ↓ | ↓ | ← | ↓ | | NT |
| 55 | ← | ↑ | ↑ | ↑ | | NT |
| 56 | ← | ↑ | ↑ | ← | | NT |
| 57 | ← | ↑ | ↑ | ↓ | C, D, J, N | PrIR-A |
| 58 | ← | ↑ | ↓ | ↑ | C, D, J, N | PrIR-A |
| 59 | ← | ↑ | ↓ | ← | C, D, J, N | PrIR-A |
| 60 | ← | ↑ | ↓ | ↓ | C, D, J, N | PrIR-A |
| 61 | ← | ↑ | ← | ↑ | | NT |
| 62 | ← | ↑ | ← | ← | | NT |
| 63 | ← | ↑ | ← | ↓ | C, D, J, N | PrIR-A |
| 64 | ← | ← | ↑ | ↑ | | NT |
| 65 | ← | ← | ↑ | ← | | NT |
| 66 | ← | ← | ↑ | ↓ | C, D, J, N | GHR-A/PrIR-A |
| 67 | ← | ← | ↓ | ↑ | C, D, J, N | GHR-A/PrIR-A |
| 68 | ← | ← | ↓ | ← | C, D, J, N | GHR-A/PrIR-A |
| 69 | ← | ← | ↓ | ↓ | C, D, J, N | GHR-A/PrIR-A |
| 70 | ← | ← | ← | ↑ | | NT |
| 71 | ← | ← | ← | ← | | NT |
| 72 | ← | ← | ← | ↓ | C, D, J, N | GHR-A/PrIR-A |
| 73 | ← | ↓ | ↑ | ↑ | | NT |
| 74 | ← | ↓ | ↑ | ← | | NT |
| 75 | ← | ↓ | ↑ | ↓ | | NT |
| 76 | ← | ↓ | ↓ | ↑ | C, D, J, N | GHR-A |
| 77 | ← | ↓ | ↓ | ← | C, D, J, N | GHR-A |
| 78 | ← | ↓ | ↓ | ↓ | C, D, J, N | GHR-A |
| 79 | ← | ↓ | ← | ↑ | | NT |
| 80 | ← | ↓ | ← | ← | | NT |
| 81 | ← | ↓ | ← | ↓ | C, D, J, N | GHR-A |

Table 1 above displays alternative expression patterns in samples obtained from tumour tissue of the polypeptides GHR, PrlR, SOCS2 and TSC2 as compared to a control sample.

↓=decreased expression

←=essentially unaltered expression

↑=increased expression compared to control tissue.

NT=Not suitable for treatment

Results obtained in the test of the present invention are subsequently correlated with table 1 and a treatment is selected based on the expression pattern. Typically—decreased expression of TSC2 and/or SOCS2 is indicative of disease when expression levels of GHR and/or PrlR are/is increased. The treatment is either by GHR antagonists, PrlR antagonists or combinations of the two antagonists. Additionally, in some embodiments the treatment may be supplemented by a dopamine agonist or the immunosupressant rapamycin.

In one embodiment the prolactin receptor antagonist of the invention is used in combination with a dopamine agonist, rapamycin or pharmaceutically active acceptable derivative thereof acting on the mTOR pathway, a GH receptor antagonist or an anti-cancer drug.

The expression levels of the above mentioned polypeptides GHR, PrlR, SOCS2 and TCS2 can be performed by any standard method well known by those of skill in the art. Routinely these methods are based on the use of antibodies such as immuno-detection systems including but not limited to Western Blot, can be used to detect specific proteins on histological section or in tissue extracts. Though not preferred, methods for determining mRNA levels of GHR, PrlR, SOCS2 and TCS2 can also be used.

In one aspect the present invention concerns a composition comprising a prolactin receptor antagonist and/or a growth hormone receptor antagonist, for use in a method of treatment of a prolactin-associated disorder in an individual, the method comprising the steps of:
a) providing a sample from tumour tissue obtained from the individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) comparing the expression levels of step b) with the expression level of a control tissue,
d) assessing a treatment regime by correlating the results of step c) with the corresponding expression pattern of table 1 above,
e) administering to the individual a therapeutically effective amount of the prolactin receptor antagonist and/or the growth hormone receptor antagonist as determined in step d).

In one aspect the invention concerns a method of treatment of a prolactin-associated disorder in an individual in need thereof, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) assessing a treatment regime,
administering to the individual a therapeutically effective amount of a composition comprising one or more prolactin receptor antagonist(s) and/or one or more growth hormone receptor antagonist(s).

In another aspect the invention concerns a method for selecting a treatment of a prolactin-associated disorder in an individual, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) selecting a treatment regime based on table 1 and/or table 2.

In one aspect the invention concerns a method for diagnosing a prolactin-associated disorder in an individual, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue, wherein an expression pattern equal to the expression patterns 3, 4, 5, 6, 9, 12, 13, 14, 15, 18, 21, 22, 23, 24, 27, 30, 31, 32, 33, 36, 39, 41, 42, 45, 57, 58, 59, 60, 63, 66, 67, 68, 69, 72, 76, 77, 78 or 81 of table 1 is indicative of a prolactin associated disorder.

The measurement of the four different biomarkers in the tumour tissue of choice, where each marker can be up (↑), down (↓) or essentially unaltered (←) compared to a normal control tissue (non-tumour tissue) renders 3⁴, i.e. the 81 different possibilities listed in table 1. However, as the method of the present invention aims to identify tumours suitable for treatment, and to guide the user of the test to select a specific treatment suitable for the specific tumour type, only a subset of the 81 alternatives results in a treatment method. Test results where both GHR and PrlR are reduced (↓) are unsuitable as are test results where TSC2 and SOCS2 are increased or normal. The test results suitable for treatment are thus restricted to those listed in table 2.

TABLE 2
Overview of expression paterns where treatment according to the present
invention is possible

| | Expression pattern | | | | | Treatment |
|---|---|---|---|---|---|---|
| No. | GHR | PrIR | SOCS2 | TCS2 | Indications | regime |
| 3 | ↑ | ↑ | ↑ | ↓ | C, D, J, N | GHR-A/PrIR-A |
| 4 | ↑ | ↑ | ↓ | ↑ | C, D, J, N | GHR-A/PrIR-A |
| 5 | ↑ | ↑ | ↓ | ← | C, D, J, N | GHR-A/PrIR-A |
| 6 | ↑ | ↑ | ↓ | ↓ | C, D, J, N | GHR-A/PrIR-A |
| 9 | ↑ | ↑ | ← | ↓ | C, D, J, N | GHR-A/PrIR-A |
| 12 | ↑ | ← | ↑ | ↓ | C, D, J, N | GHR-A |
| 13 | ↑ | ← | ↓ | ↑ | C, D, J, N | GHR-A |
| 14 | ↑ | ← | ↓ | ← | C, D, J, N | GHR-A |
| 15 | ↑ | ← | ↓ | ↓ | C, D, J, N | GHR-A |
| 18 | ↑ | ← | ← | ↓ | C, D, J, N | GHR-A |
| 21 | ↑ | ↓ | ↑ | ↓ | C, D, J, N | GHR-A |
| 22 | ↑ | ↓ | ↓ | ↑ | C, D, J, N | GHR-A |
| 23 | ↑ | ↓ | ↓ | ← | C, D, J, N | GHR-A |
| 24 | ↑ | ↓ | ↓ | ↓ | C, D, J, N | GHR-A |
| 27 | ↑ | ↓ | ← | ↓ | C, D, J, N | GHR-A |
| 30 | ↓ | ↑ | ↑ | ↓ | C, D, J, N | PrIR-A |
| 31 | ↓ | ↑ | ↓ | ↑ | C, D, J, N | PrIR-A |
| 32 | ↓ | ↑ | ↓ | ← | C, D, J, N | PrIR-A |
| 33 | ↓ | ↑ | ↓ | ↓ | C, D, J, N | PrIR-A |
| 36 | ↓ | ↑ | ← | ↓ | C, D, J, N | PrIR-A |
| 39 | ↓ | ← | ↑ | ↓ | C, D, J, N | PrIR-A |
| 41 | ↓ | ← | ↓ | ← | C, D, J, N | PrIR-A |
| 42 | ↓ | ← | ↓ | ↓ | C, D, J, N | PrIR-A |
| 45 | ↓ | ← | ← | ↓ | C, D, J, N | PrIR-A |
| 57 | ← | ↑ | ↑ | ↓ | C, D, J, N | PrIR-A |
| 58 | ← | ↑ | ↓ | ↑ | C, D, J, N | PrIR-A |
| 59 | ← | ↑ | ↓ | ← | C, D, J, N | PrIR-A |
| 60 | ← | ↑ | ↓ | ↓ | C, D, J, N | PrIR-A |
| 63 | ← | ↑ | ← | ↓ | C, D, J, N | PrIR-A |
| 66 | ← | ← | ↑ | ↓ | C, D, J, N | GHR-A/PrIR-A |
| 67 | ← | ← | ↓ | ↑ | C, D, J, N | GHR-A/PrIR-A |
| 68 | ← | ← | ↓ | ← | C, D, J, N | GHR-A/PrIR-A |
| 69 | ← | ← | ↓ | ↓ | C, D, J, N | GHR-A/PrIR-A |
| 72 | ← | ← | ← | ↓ | C, D, J, N | GHR-A/PrIR-A |
| 76 | ← | ↓ | ↓ | ↑ | C, D, J, N | GHR-A |
| 77 | ← | ↓ | ↓ | ← | C, D, J, N | GHR-A |
| 78 | ← | ↓ | ↓ | ↓ | C, D, J, N | GHR-A |
| 81 | ← | ↓ | ← | ↓ | C, D, J, N | GHR-A |

The method according to the present invention is suitable for selecting treatment of prolactin associated disorders selected from the group consisting of colon cancer, liver cancer, breast cancer, endometrial cancer, ovary cancer, prostate cancer, parathyroid cancer, benign breast tumour, leiomyoma, renal angiomyolipoma, acromegaly, hyperprolactinemia, obesity resulting from endocrine malfunction, lymphangioleiomyomatosis, lupus erythematosus, benign prostate tumour and peripartum cardiomyopathy.

According to one aspect of the invention the prolactin-related disease is dopamine resistant hyperprolactinemia, drug induced hyper prolactinemia, conditions involving changed Prl sensitivity, lympangioleomyomatosis, tuberous sclerosis and conditions characterized by overproduction of Prl.

The arrow down (↓) in tables 1 and 2 indicates decreased expression compared to control tissue, while arrow left (←) indicates essentially unaltered expression compared to control tissue, and arrow up (↑) indicates increased expression compared to control tissue. GHR-A is a Growth Hormone Receptor Antagonist, and PrlR-A is a Prolactin

Receptor Antagonist. GHR-A/PrlR-A is a combination treatment of a Growth Hormone Receptor Antagonist and a Prolactin Receptor Antagonist.

Biomarkers It is an object of the present invention to determine expression patterns of the polypeptides PrlR, GHR, SOCS2 and TCS2 (also referred to as the biomarkers of the invention) and to determine a treatment regime based on said expression pattern.

The growth hormone receptor (GHR) detected in the method of the present invention typically has the sequence of SEQ ID NO: 1 or a variant thereof being at least 70% identical to SEQ ID NO: 1.

The prolactin receptor (PrlR) detected in the method of the present invention typically has the sequence of SEQ ID NO: 2 or a variant thereof being at least 70% identical to SEQ ID NO: 2.

The suppressor of cytokine signalling 2 (SOCS2) detected in the method of the present invention typically has the sequence of SEQ ID NO: 3 or a variant thereof being at least 70% identical to SEQ ID NO: 3.

The tuberous sclerosis complex 2 (TSC2) detected in the method of the present invention typically has the sequence of SEQ ID NO: 4 or a variant thereof being at least 70% identical to SEQ ID NO: 4.

The Prl and GH receptor antagonists can be any antagonist capable of functionally blocking signalling through said receptors.

It is thus an aspect of the present invention to provide prolactin receptor antagonists i.e. compounds blocking the prolactin receptor. Principally such antagonists can be based on the backbone of native prolactin protein where one or more substitutions/mutations of certain amino acids can convert the prolactin receptor agonist prolactin into a prolactin receptor antagonist.

It is an aspect of the present invention to provide the use of certain Prl variants with high affinity for the Prl receptor to create antagonists that prevent prolactin receptor dimerization.

According to a further aspect of the invention, a diagnostic procedure can be carried out to determine the sensitivity of a tumour for the above said antagonists and in this procedure tumour material are tested for the following proteins; GH receptor, Prl receptor, SOCS2 and TSC2.

Agents of the Invention

Mature human wild type prolactin (Prl) is a single chain polypeptide of 199 amino acid residues with a molecular weight of about 24,000 Daltons. It is synthesised in the adenohypophysis (anterior pituitary gland), in the breast and in the decidua and has a structure similar to that of growth hormone (GH) and placental lactogen (PL). The molecule is folded due to the activity of three disulfide bonds. The sequence of human prolactin is given in SEQ ID NO. 6.

Human prolactin (Prl) has two separate and different binding sites (site 1 and site 2) that each interact with a prolactin receptor to form a 1:2 ligand-receptor complex. The classical model (Fuh, G., Cunningham, B. C, Fukunaga, R., Nagata, S., Goeddel, D. V., and Wells, J. A. (1992) Science 256, 1677-1680) for activation of the Prl and GH receptors (PrlR and GHR, respectively) describes the signaling molecular entity as a ternary complex between one hormone molecule and a receptor homo-dimer assembled in a strictly sequential and hormone dependent fashion: first the hormone ligand engages via site 1 in high affinity binding to one receptor chain forming a 1:1 hormone/receptor complex, which constitutes the template for binding a second, identical receptor molecule resulting in the active 1:2 complex. However, this model has been challenged by an increasing body of experimental evidence. Initially reported for the homologous human erythropoietin receptor (hEPOR) (Constantinescu, S. N., Keren, T., Socolovsky, M., Nam, H. S., Henis, Y. I., and Lodish, H. F. (2001) Proceedings of the National Academy of Sciences of the United States of America 9JS14379-4384) and later for hGHR (Brown, R. J., Adams, J. J., Pelekanos, R. A., Wan, Y., McKinstry, W. J., Palethorpe, K., Seeber, R. M., Monks, T. A., Eidne, K. A., Parker, M. W., and Waters, M. J. (2005) Nature Structural & Molecular Biology Î 814-821) and PrlR (Qazi, A. M., Tsai-Morris, C. H., and Dufau, M. L. (2006) Molecular Endocrinology 20, 1912-1923), these studies support that preformed, inactive dimers exist in the absence of hormone. This infers that receptor dimerization is a necessary, but not sufficient, event for receptor activation, and, notably, not a strictly ligand dependent one. For both hEPOR (Seubert, N., Royer, Y., Staerk, J., Kubatzky, K. F., Moucadel, V., Krishnakumar, S., Smith, S. O., and Constantinescu, S. N. (2003) Molecular Cell Ylx 1239-1250) and hGHR (Brown, R. J., Adams, J. J., Pelekanos, R. A., Wan, Y., McKinstry, W. J., Palethorpe, K., Seeber, R. M., Monks, T. A., Eidne, K. A., Parker, M. W., and Waters, M. J. (2005) Nature Structural & Molecular Biology 1̂. 814-821) models have been proposed, where receptor activation involves relative rotations and movements of receptor subunits induced by hormone binding. Allosteric reorganization of the intracellular receptor domains brings about activation of the receptor associated Janus family of tyrosine kinases JAK2 or JAK1, which stimulate signal transducers and activators of transcription STAT5 or STAT3, respectively. Receptor activation also leads to the activation of Ras/Raf/MAPK kinase/Erk and phosphatidylinositol 3-kinase/Akt signalling pathways. It is primarily via these pathways that the receptors for these ligands induce cell differentiation, proliferation, and/or survival (Ihle, J. N. and Kerr, I. M. (1995) Trends in Genetics IL 69-74)

The present invention concern molecules capable of acting as antagonists of the prolactin receptor (PrlR) thus being prolactin receptor antagonists (PrlR-A). The term “prolactin receptor antagonist” as used herein thus refers to a ligand of the prolactin receptor having antagonistic activity of said prolactin receptor, causing it to act as an inhibitor of one or more cellular processes. Such prolactin antagonistic activity may be measured by Western blot analysis of the phosphorylation status of STAT5 as set out in Langenheim, J. F. et al, Mol Endocrinol, 2006; 20(39):661-674. Without being bound by theory, it is believed that a prolactin receptor ligand, that comprises one or more mutations that affect the structural integrity of ‘Site 2’, will not trigger the receptor, and activate subsequent signal transduction, because it can not productively interact with the second receptor chain. Thus, such a ligand does not activate the receptor and instead acts as prolactin receptor antagonist. Six prolactin receptor antagonists are currently known in the literature (Goffin et al. Endocrine Rev. 2005, 26, 400-422):

(a) G120R/K-hGH, a variant of human growth hormone;

(b) G120R-hPL, a variant of human placental lactogen;

(c) G129R-Prl a full-length variant of human prolactin;

(d) 5179D-Prl, a full-length variant of human prolactin;

(e) G129R-Prl (10-199), a truncated variant of human prolactin; and

(f) G129R-Prl (15-199), a truncated variant of human prolactin.

In one aspect, the present invention is directed to a composition capable of binding to and antagonizing the prolactin receptor (PrlR) and/or the growth hormone receptor (GHR).

In one embodiment the composition comprises one or more of the above mentioned antagonists.

In one embodiment the composition is a PrlR antagonist selected from the group consisting of SEQ ID NO: 7 and 8 and truncated derivatives thereof such as Prl Δ1-n S33A, Q73L, G129R, K190R and Prl Δ1-n S61A, D68N, Q73L, G129R, K190R wherein n=1, 2, 3, 5, 5, 6, 7, 8 or 9, and in particular n=9, such as SEQ ID NOs: 16, 17, 18, 19, 20, 21, 22, 23, 24, 25, 26, 27, 28, 29, 30, 31, 32, 32 and 33 are high affinity Prl receptor antagonists with unexpected advantages for treatment of certain Prl related medical conditions, in particular with, but not limited to, dopamine resistant hyperprolactinemia, drug induced hyper prolactinemia, conditions involving changed Prl sensitivity, lympangioleomyomatosis, tuberous sclerosis and conditions characterized by overproduction of Ph, e.g. prostate cancer, ductal prostate cancer, liver cancer and myomas and endometrial cancer. Conditions associated with metabolic abnormalities e.g. obesity can also react to treatment with prolactin receptor antagonists.

The present inventor has identified specific intracellular proteins that regulate Prl receptor levels. We disclose that altered levels of these intracellular proteins increase levels of Prl receptors thereby causing a hypersensitive state and such hypersensitive states are suitable for treatment with Prl receptor antagonists. A prolactin hypersensitive state is expected to react to a prolactin receptor antagonist by reduction of growth—the reasons for this could be changes levels of growth stimulatory effector molecular but also changes metabolism of the cell. The said intracellular regulators can be divided into two categories: one is involved in regulation of protein synthesis and another in the regulation of protein break down.

The molecular nature of these intracellular regulators is disclosed and exemplified as tubero sclerosis complex/tuberin (TSC) and suppressor of cytokine signaling (SOCS). Both TSC (notably TSC2) and SOCS (notably SOCS2 and SOCS3) are related to human disorders and disorders that involve reduced TSC and/or SOCS are associated with a hypersensitive state for prolactin that can be treated using Prl receptor antagonists. The group of TSC related disorders comprises lymphangioleiomyomatosis and tuberosclerosis, and as TSC suppress the mTOR pathway, the alteration of the mTOR pathway in several hyperproliferative disorder, e.g. breast cancer and prostate cancer, is also of importance in this connection.

Deficiency of SOCS will lead to excessive STAT activation and such elevation is frequently seen in certain tumours, e.g. myoma/leiomyoma, prostate cancer, breast cancer, liver cancer, ovary cancer and pancreas cancer.

In one embodiment the composition according to the invention is selected from the group consisting of:
a) an isolated polypeptide comprising:
   i) an amino acid sequence selected from the group consisting of SEQ ID NOs: 7, 8, 16, 17, 18, 19, 20, 21, 22, 23, 24, 25, 26, 27, 28, 29, 30, 31, 32, 32 and 33; or
   ii) a biologically active sequence variant of the amino acid sequence of i) wherein the variant has at least 70% sequence identity to said SEQ ID NO: 7, 8, 16, 17, 18, 19, 20, 21, 22, 23, 24, 25, 26, 27, 28, 29, 30, 31, 32, 32 and 33,
   iii) a biologically active fragment of at least 15 contiguous amino acids of any one of i) through ii), said fragment having at least 70% sequence identity to SEQ ID NO: 7, 8, 16, 17, 18, 19, 20, 21, 22, 23, 24, 25, 26, 27, 28, 29, 30, 31, 32, 32 or 33 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

A major issue in the development of protein based pharmaceutical drugs is the stability of the protein from its production to its final administration, and major efforts are typically put in to identification of a formulation that provides adequate physical and chemical stability of the drug substance. Chemical instability of proteins is mainly associated with oxidative or hydrolytic modifications at sensitive residues in the amino acid sequence. For example, the side chain amide group in glutaminyl or asparaginyl residues may be hydrolysed to form a free carboxylic acid in a process called deamidation. This can lead to the formation of chemical degradation products with altered and potentially undesired biological properties. The rate of deamidation is strongly dependent on several physical and chemical conditions, particularly temperature, pH, dielectric constant of solvent but also the presence of certain ions, such as phosphate which has been shown to catalyze the Asn deamidation reactions. Furthermore, both the primary amino acid sequence and secondary structure strongly influence the rate of deamidation (Robinson, N. E., Robinson, Z. W., Robinson, B. R., Robinson, A. L., Robinson, J. A., Robinson, M. L., and Robinson, A. B. (2004) Journal of Peptide Research 63, 426-436). In the primary sequence particularly the nature of the amino acid immediately after (position n+1) influences the deamidation rate of an Asn-residue (position n). Asn-residues followed in the amino acid sequence by a Glycine are particularly susceptible to deamidation. The rate of deamidation for an Asn-residue (position n) is to a lesser extent influenced by the nature of amino acid in position n−1. Asn residues situated in loosely structured and solvent exposed peptide segments are typically more susceptible to deamidation as compared to Asn-residues situated in secondary structure elements (helix and sheet).

Prl has been reported to readily undergo deamidation, and deamidated Prl-variants have been encountered in most species examined, including mouse, rat, sheep and human (Sinha, Y. N. (1995) Endocrine Reviews 354-369). Deamidation of Prl has been shown to alter its biological properties. Thus, reduced potency were reported for deamidated forms of rat and mouse Prl (Haro, L. S. and Talamantes, F. J. (1985) Endocrinology 116, 353-358, Sinha, Y. N. and Gilligan, T. A. (1981) Endocrinology 108, 1091-1094). Nyberg et al. (Nyberg, F., Roos, P., and Wide, L. (1980) Biochimica et Biophysica Acta 625, 255-265) separated human pituitary Prl in to separate isoforms differing in their net charges and attributed to deamidated Prl-species. The deamidated isoforms of Prl were shown to possess similar or even slightly increased lactogenic activity as measured by the pigeon crop-sac assay.

In one embodiment, the prolactin or prolactin analogue comprises a mutation N56X of SEQ ID NO. 6, wherein X is an amino acid residue which is resistant to deamidation. It will be appreciated that the mutation does not significantly impair the binding of the ligand to the receptor, e.g. the mutated prolactin or prolactin analogue may for instance have at least 25%, such as at least 50%, for instance at least 60% of the binding affinity for the prolactin receptor of the unmutated prolactin or prolactin analogue, but the precise ratio is not that important as long as the prolactin analogue can bind the prolactin receptor efficiently, even prolactin analogues with moderately reduced binding affinity, but which are resistant to deamidation, are of interest. As an example from the related erythropoietin (Epo) system, a hyperglycosylated analog of Epo, called novel erythropoiesis-stimulating protein (NESP), has a lower affinity than Epo for the Epo receptor but has greater in vivo activity and a longer serum half-life than Epo (Gross, A. W. and Lodish, H. F. Journal of Biological Chemistry 281, 2024-2032 (2006)) In a further embodiment, X is glycine.

In one embodiment, the prolactin receptor antagonist comprises a mutation S57Y of SEQ ID NO. 6, wherein Y is an amino acid residue that suppresses deamidation of the amino acid residue in position 56 of SEQ ID NO. 6. In one embodiment, Y is any of the natural amino acids encoded by the genetic code other than serine and glycine. In one embodiment, Y is selected from the amino acids valine, leucine, isoleucine, tryptophan, tyrosine, phenylalanine, proline and threonine.

In one embodiment, the prolactin or prolactin analogue comprises a mutation 155Z, wherein Z is an amino acid residue that suppresses deamidation of the amino acid residue in position 56 of SEQ ID NO. 6. In one embodiment, Z is any of the natural amino acids encoded by the genetic code other than isoleucine. In one embodiment, Z is any of the amino acids comprising valine, leucine, tryptophan, tyrosine, phenylalanine, proline and threonine.

Without being bound by theory, it is believed that N56 of prolactin makes contact with the receptor, and thus that deamidation of N56 reduces affinity for the receptor. Thus, the mutations as hereinbefore defined help to maintain the activity of prolactin or a prolactin analogue by increasing the chemical stability of prolactin or a prolactin analogue in respect of deamidation.

This is important from a manufacturing point of view, because the production and purification processes will be complicated by the formation of deamidated products. Furthermore, stabilized proteins will increase the shelf life of the final product and improve overall economy. Finally, increased stability is also expected to be correlated with improved pharmacokinetic properties of the protein.

The present invention also provides a method for stabilising prolactin or a prolactin analogue, which method comprises mutating one or more amino acids in a position resulting in induction of an altered secondary structure at a position corresponding to positions 55, 56 or 57 of SEQ ID NO. 6.

The present invention also provides a method for stabilising prolactin or a prolactin analogue, which method comprises mutating one or more amino acids in a position resulting in induction of an altered secondary structure at a position corresponding to positions 55 or 57 of SEQ ID NO. 6. In one embodiment, the method comprises mutating one or more amino acids in the segment corresponding to positions 47-57 of SEQ ID NO. 6.

In one embodiment, the method comprises mutating one or more amino acids corresponding to positions 55, 56 or 57 of SEQ ID NO. 6. The present invention also provides a method for stabilising prolactin or a prolactin analogue comprising substituting asparagine in position 56 of SEQ ID NO. 6 with an amino acid residue which is resistant to deamidation.

In a further embodiment, the method comprises substituting asparagine in position 56 of SEQ ID NO. 6 with glycine. In a further embodiment, the method comprises substituting asparagine in position 56 of SEQ ID NO. 6 with glutamine.

The present invention also provides a method for stabilising prolactin or a prolactin analogue comprising substituting the amino acid residue in the position corresponding to position 55 (isoleucine in wtPrl) or the amino acid residue in the position corresponding to position 57 (serine in wtPrl) of SEQ ID NO. 6 with an amino acid residue which suppresses deamidation of the amino acid residue in position 56 of SEQ ID No. 6. In one embodiment, the method comprises substituting the amino acid in position 55 of SEQ ID NO. 6 with an amino acid residue which is any of the 20 amino acids other than glycine and isoleucine and/or substituting the amino acid in position 57 of SEQ ID NO. 6 with an amino acid residue which is any of the 20 amino acids other than serine and glycine.

In another embodiment the composition is selected from the group consisting of:
a) an isolated polypeptide comprising:
   i) the amino acid sequence of SEQ ID NOs: 10; or
   ii) a biologically active sequence variant of the amino acid sequence of i) wherein the variant has at least 70% sequence identity to said SEQ ID NO: 10,
   iii) a biologically active fragment of at least 15 contiguous amino acids of any one of i) through ii), said fragment having at least 70% sequence identity to SEQ ID NO: 10 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In one embodiment the composition as defined herein is a polypeptide wherein the polypeptide is a naturally occurring allelic variant of a sequence selected from the group consisting of SEQ ID NOs: 7, 8, 16, 17, 18, 19, 20, 21, 22, 23, 24, 25, 26, 27, 28, 29, 30, 31, 32, 32, 33 and 10.

In one embodiment the composition is a polypeptide variant, wherein any amino acid specified in the selected sequence is altered to provide a conservative substitution, with the proviso that no more than 50 amino acids are so altered.

In one embodiment the composition is a polypeptide variant, wherein the polypeptide is a variant polypeptide described therein, wherein any amino acid specified in the selected sequence is altered to provide a conservative substitution, with the proviso that no more than 25 amino acids are so altered.

Preferably the composition according to the invention is a polypeptide having at least 65%, more preferably at least 70%, more preferably at least 75%, preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% sequence identity to an amino acid sequence selected from the group consisting of SEQ ID NOs: 7, 8, 16, 17, 18, 19, 20, 21, 22, 23, 24, 25, 26, 27, 28, 29, 30, 31, 32, 32, 33 and 10.

In one embodiment the growth hormone receptor antagonist (GHR-A) is a stable sequence variant of SEQ ID NO. 9, wherein said sequence variant comprises at least one amino acid residue which has been altered to a different amino acid residue.

Typically the prolactin receptor antagonist (PrlR-A) polypeptide of the invention is a polypeptide derived from wild-type prolactin, and modified to obtain a sequence variant of prolactin having suitable prolactin receptor antagonising properties. In one embodiment the sequence variant is less than 99% identical to SEQ ID NO. 6, preferably less than 98% identical to SEQ ID NO. 6, preferably less than 97% identical to SEQ ID NO. 6, preferably less than 96% identical to SEQ ID NO. 6, preferably less than 95% identical to SEQ ID NO. 6, preferably less than 94% identical to SEQ ID NO. 6, preferably less than 93% identical to SEQ ID NO. 6, preferably less than 92% identical to SEQ ID NO. 6, preferably less than 91% identical to SEQ ID NO. 6, preferably less than 90% identical to SEQ ID NO. 6, preferably less than 85% identical to SEQ ID NO. 6, preferably less than 80% identical to SEQ ID NO. 6, preferably less than 75% identical to SEQ ID NO. 6, preferably less than 70% identical to SEQ ID NO. 6, with the proviso that the sequence variant of SEQ ID NO. 6 is capable of binding to and inhibiting cellular signaling through the prolactin receptor.

In one embodiment the prolactin receptor antagonist (PrlR-A) is a stable sequence variant of SEQ ID NO. 6 wherein said sequence variant comprises at least one amino acid residue which has been altered to a different amino acid residue.

In one embodiment the PrlR-A is a sequence variant less than 99% identical to SEQ ID NO. 5, e.g. less than 98% identical to SEQ ID NO. 5, e.g. less than 97% identical to SEQ ID NO. 5, e.g. less than 96% identical to SEQ ID NO. 5, e.g. less than 95% identical to SEQ ID NO. 5, e.g. less than 94% identical to SEQ ID NO. 5, e.g. less than 93% identical to SEQ ID NO. 5, e.g. less than 92% identical to SEQ ID NO. 5, e.g. less than 91% identical to SEQ ID NO. 5, e.g. less than 90% identical to SEQ ID NO. 5, e.g. less than 85% identical to SEQ ID NO. 5, e.g. less than 80% identical to SEQ ID NO. 5, e.g. less than 75% identical to SEQ ID NO. 5, e.g. less than 70% identical to SEQ ID NO. 5, with the proviso that the sequence variant of SEQ ID NO:5 is capable of binding to and inhibiting cellular signaling through the prolactin receptor.

In one embodiment, the PrlR-A is a Prl variant mutated in one or more of the positions corresponding to positions 61, 71 and/or 73 of SEQ ID NO. 6.

The antagonist of the invention may be a prolactin receptor antagonist selected from anti-prolactin receptor antibodies or an antibody fragments thereof.

The antagonist of the invention may also be a growth hormone receptor antagonist selected from anti-growth hormone receptor antibodies or an antibody fragments thereof.

The anti-prolactin receptor antibodies and anti-growth hormone receptor antibodies of the invention are selected from monoclonal antibodies and polyclonal antibodies.

In certain embodiments it is advantageous to co-administer a further active ingredient along with, or sequentially with, the PrlR-A and/or GHR-A of the invention. The further active ingredient is in one embodiment selected from the group consisting of a dopamine agonist, rapamycin or a derivative of any one of the dopamine agonist or rapamycin, wherein the derivative is capable of acting on the mTOR pathway, a GH receptor antagonist or an anti-cancer drug.

It is also an object of the invention to provide fragments of prolactin and growth hormone, In particular sequence variants being fragments of prolactin and growth hormone. Thus in one embodiment the composition of the invention is a biologically active polypeptide fragment, wherein the fragment comprises less than 199 contiguous amino acid residues, such as less than 198 contiguous amino acid residues, for example less than 197 contiguous amino acid residues, such as less than 196 contiguous amino acid residues, for example less than 195 contiguous amino acid residues, such as less than 194 contiguous amino acid residues, for example less than 193 contiguous amino acid residues, such as less than 192 contiguous amino acid residues, for example less than 191 contiguous amino acid residues, such as less than 190 contiguous amino acid residues, for example less than 189 contiguous amino acid residues, such as less than 188 contiguous amino acid residues, for example less than 187 contiguous amino acid residues, such as less than 186 contiguous amino acid residues, for example less than 185 contiguous amino acid residues, such as less than 184 contiguous amino acid residues, for example less than 183 contiguous amino acid residues, such as less than 182 contiguous amino acid residues, for example less than 181 contiguous amino acid residues, for example less than 180 contiguous amino acid residues, such as less than 160 contiguous amino acid residues, for example less than 150 contiguous amino acid residues, such as less than 140 contiguous amino acid residues, for example less than 130 contiguous amino acid residues, such as less than 120 contiguous amino acid residues, for example less than 110 contiguous amino acid residues, such as less than 100 contiguous amino acid residues, for example less than 90 contiguous amino acid residues, such as less than 85 contiguous amino acid residues, for example less than 80 contiguous amino acid residues, such as less than 75 contiguous amino acid residues, for example less than 70 contiguous amino acid residues, such as less than 65 contiguous amino acid residues, for example less than 60 contiguous amino acid residues, such as less than 55 contiguous amino acid residues, for example less than 50 contiguous amino acid residues, such as less than 45 contiguous amino acid residues, for example less than 40 contiguous amino acid residues, such as 35 contiguous amino acid residues, for example 30 contiguous amino acid residues, such as 25 contiguous amino acid residues, such as 20 contiguous amino acid residues, for example 15 contiguous amino acid residues of an any one of the amino acid sequences selected from the group consisting of SEQ ID NOs: 7, 8, 16, 17, 18, 19, 20, 21, 22, 23, 24, 25, 26, 27, 28, 29, 30, 31, 32, 32, 33 and 10.

In one embodiment the composition is a biologically active fragment of a polypeptide as defined herein, wherein the fragment comprises at least 15 contiguous amino acid residues, such as more than 20 contiguous amino acid residues, for example more than 25 contiguous amino acid residues, for example more than 50 contiguous amino acid residues, such as more than 75 contiguous amino acid residues, for example more than 100 contiguous amino acid residues, such as more than 125 contiguous amino acid residues, for example more than 150 contiguous amino acid residues, such as more than 175 contiguous amino acid residues of any one of the amino acid sequences selected from the group consisting of SEQ ID NOs: 7, 8, 16, 17, 18, 19, 20, 21, 22, 23, 24, 25, 26, 27, 28, 29, 30, 31, 32, 32, 33 and 10.

Any one of the polypeptides and fragments and variants thereof disclosed herein may be glycosylated, either spontaneously or by chemical or biochemical modification. Thus in one embodiment the composition according to the invention is a polypeptide which is glycosylated.

Polypeptides comprising cysteine residues are capable of forming intra and/or intermolecular cystin (S—S) bridges. Thus in one embodiment the composition according to the invention is capable of forming at least one intramolecular cystin bridge.

In one embodiment the composition is a polypeptide which comprises a dimer of said polypeptide linked through at least one intermolecular cystin bridge.

The polypeptide being a PrlR-antagonist or a GHR antagonist can be modified by molecular biological methods well known by those of skill in the art, to alter biophysical properties of the polypeptide or to facilitate purification, e.g. by affinity chromatography. Accordingly in one embodiment the composition is a polypeptide further comprising an affinity tag, such as a polyhis tag, a GST tag, a HA tag, a Flag tag, a C-myc tag, a HSV tag, a V5 tag, a maltose binding protein tag, a cellulose binding domain tag.

One important biophysical property that is altered in an embodiment of the present invention is the composition's degradation rate. In one embodiment the PrlR-A polypeptide and/or the GHR-A polypeptide is thus chemically modified in order to increase its half-life when administered to a patient, in particular its plasma half-life.

A number of methods are available in the art for modification of peptide drugs in order to increase its half-life, and such methods of the art can be employed for modification of the PrlR antagonists and GHR antagonists being polypeptides of the present invention, including fragments and variants thereof. Short plasma half-life times are often caused by fast renal clearance as well as enzymatic degradation occurring during systemic circulation. Modifications of the peptide/protein can lead to prolonged plasma half-life times. Increased half-life can for example be obtained by shortening the overall amino acid amount of the polypeptide.

Exopeptidases is a prominent group of proteolytic enzymes occurring in plasma, liver and kidney, which affect therapeutic peptides and proteins. Thus, modification of eitherone, or both of the peptide drug termini in many cases increase enzymatic stability, and thus plasma half-life. Thus, in one approach, one or more additional compounds are coupled to a polypeptide of the present invention, in order to increase its plasma half-life. In one embodiment, the terminal modification is N-acetylation and/or C-amidation. In another such embodiment, The N and/or C-terminus is conjugated to polyethylenglycol (PEG) compounds. One specific modification of the polypeptide is the dual modification of N-terminal palmitoyl and C-terminal PEGylation. A headto-tail cyclization of the polypeptide drug by the formation of an amide bond between C- and N-terminus is also possible in order to prevent exopeptidase caused degradation of the PrlR-A and GHR-A polypeptides.

In another embodiment, increased plasma half-life is obtained by replacement of one or more amino acids, which are known to be susceptible to enzymatic cleavage, thereby letting the polypeptide escape proteolytic degradation. For example, one or more L-amino acids could be substituted with D-amino acids at one or both polypeptide termini, and/or within the polypeptide in order to avoid degradation, and thereby increase plasms half-life.

Increased half-life of the polypeptide of the invention can also be obtained by coadministration of the polypeptide with one or more specific enzyme inhibitors. Such enzyme inhibitors could be included in the kit-of-parts of the invention. In yet another approach, increased half-life could be obtained by increasing the molecular mass of the PrlR-A and/or GHR-A polypeptide of the invention.

As a general rule, substances with a molecular mass below 5 kDa which are not bound to plasma proteins are excreted via the renal route, whereas molecules with a molecular mass over 50 kDa cannot or only in very small amounts be found in the glomerular ultrafiltrate. Accordingly, a main reason for short peptide and protein half-life time beside enzymatic degradation is their fast renal excretion. Therefore, half-life time can be prolonged by increasing the polypeptide drug size. Furthermore, a synergistic effect may be given by additional enzyme inhibition. Beside chemical modification of N- and C-termini which is an effective way to inhibit exopeptidases and replacement of labile amino acids, PEGylation allows to specifically protect endangered termini and furthermore increases molecular mass. In addition, PEGylation within the drug molecule expectedly leads to improved enzymatic stability mediated by a steric hindrance of proteolytic enzymes.

Poly(ethyleneglycol) (PEG) exhibits several beneficial properties: high water solubility, high mobility in solution, lack of toxicity and immunogenicity and ready clearance from the body. Very often these properties are transferred to PEG-protein or PEG-peptide conjugates. The extent of these feature are dependent on the molecular weight of the attached PEG.

Also polymers of N-acetylneuraminic acid (polysialic acids) may be used as conjugates to a polypeptide of the invention. Polysialic acids are naturally occurring, biodegradable, highly hydrophilic compounds which have no known receptors in the human body. PEGylation and sialyation prolong half-life time by a combination of two mechanisms—improvement of enzymatic stability and decrease of renal excretion by increasing molecular mass.

Albumin is known to have a long plasma half-life and because of this property it has been used in drug delivery in order to increase half-life of drugs. For this purpose albumin has been conjugated to such pharmaceutical compounds. Especially suitable is coupling to the free cysteine residue on the albumin molecule (Cys 34), e.g. by methods described in WO2010/092135, especially the methods using PDPH (3-(2-pyridyldithio) propionyl hydrazide) to link albumin to a PrlR-A and/or GHR-A polypeptide of the invention including fragments thereof via a hydrazone link to the PrlR-A and/or GHR-A polypeptide. Another coupling technology is described by Neose (see eg US2004/0126838) using enzymatic glycoconjugation. This technology can be used to link e.g. albumin to a PrlR-A and/or GHR-A polypetide of the invention using a suitable linker.

In certain embodiments the present invention concerns the use of a long-acting modified PrlR-A and/or GHR-A polypeptide, wherein said modified polypeptide comprises a mammalian PrlR-A and/or GHR-A or analog thereof linked to a pharmaceutically acceptable molecule, e.g. human PrlR-A and/or GHR-A linked to, e.g. fused to, albumin, or fused to a fatty acid of suitable length, or fused to an Fc fragment of a mammalian antibody, or a variant of an Fc fragment of a mammalian antibody or conjugated to an acylation group or PEG, that in some embodiments provides an in vivo plasma half-life of the mammalian PrlR-A and/or GHR-A or analog thereof, or the modified PrlR-A and/or GHR-A which is from 2 to 48 hours or longer, typically from 4 to 28 hours, such as 6-8 hours in a mammal. A long acting GH has previously been described in [12].

The creation of fusion proteins comprised of immunoglobulin constant regions linked to a protein of interest, or fragment thereof, has been described (see, e.g., U.S. Pat. Nos. 5,155,027, 5,428,130, 5,480,981, and 5,808,029). These molecules usually possess both the biological activity associated with the linked molecule of interest as well as the effector function, or some other desired characteristic, associated with the immunoglobulin constant region. Fusion proteins comprising an Fc portion of an immunoglobulin can bestow several desirable properties on a fusion protein including increased stability, increased serum half-life (see Capon et al. (1989) Nature 337:525) as well as binding to Fc receptors such as the neonatal Fc receptor (FcRn) (U.S. Pat. Nos. 6,086,875, 6,030,613, and 6,485,726).

In one embodiment the moiety resulting in increased half-life is a multifunctional moiety, such as bi- or trifunctional, which may be covalently linked to one or more PrlR-A and/or GHR-A molecules, such as one or more mammalian PrlR-A and/or GHR-A molecule, and covalently linked to one or more pharmaceutically acceptable molecule(s) so as to create the modified PrlR-A and/or GHR-A compound. The linker may be stabile which means that no significant chemical reactions, e.g. hydrolysis, occurs at physiological conditions (e.g. temperature of 37° C. and pH 7.4) over the time period of the treatment. This can be determined by stability studies known in the art. The linker may be a chemical linker meaning that it is generated by organic chemistry outside a living cell. The linker may be a sugar moiety, such as a glycosylation on a protein, or may be chemically prepared and used to link the PrlR-A and/or GHR-A molecule, and a second pharmaceutically acceptable molecule such as PEG variants, albumin, fatty acids or antibodies or antibody fragments such as Fc fragments.

In one embodiment, the composition, such as PrlR-A and/or GHR-A polypetide, of the invention is coupled to a immunoglobulin-Fc such as IgG-Fc.

The PrlR-A and/or GHR-A compound of the present invention may optionally comprise at least one peptide linker. In one embodiment, the linker is comprised of amino acids linked together by peptide bonds, wherein the amino acids are selected from the twenty naturally occurring amino acids. In various embodiments the linker can comprise 1-5 amino acids, 1-10 amino acids, 1-20 amino acids, 10-50 amino acids, 50-100 amino acids, or 100-200 amino acids. In one embodiment the amino acids are selected from glycine, alanine, proline, asparagine, glutamine, and lysine. In one embodiment a linker is made up of a majority of amino acids that are sterically unhindered, such as glycine and alanine. The linker in one embodiment can comprise the sequence Gn (equivalently, -(Gly)n-). The linker can in one embodiment comprise the sequence (GGS)n or (GGGGS)n. In each instance, n is an integer, such as 1, 2, 3, 4, 5, 6, 7, 8, 9, or 10. Examples of linkers include, but are not limited to, GGG, SGGSGGS (SEQ ID NO:11), GGSGGSGGSGGSGGG (SEQ ID NO:12), GGSGGSGGSGGSGGSGGS (SEQ ID NO:13), GGGGSGGGGSGGGGS (SEQ ID NO:14) and EFAGAAAV (SEQ ID NO:15).

In one embodiment the peptide linker has at least 1 amino acid, such as from 1-200 amino acids, typically 1-50 amino acids wherein the amino acids are selected from the twenty naturally occurring amino acids. Typically, the peptide linker has from 1-40 amino acids, such as from 1-30, such as from 1-20, such as from 1-10 amino acids. In a further embodiment the peptide linker is selected from a linker made up of amino acids selected from glycine, alanine, proline, asparagine, glutamine, and lysine. Typically, the peptide linker is made up of a majority of amino acids that are sterically unhindered, such as glycine and alanine. In particular, the peptide linker comprises a sequence selected from -(G)n-, (GGS)n or (GGGGS)n, wherein n is an integer of from 1-50. Typically n is an integer selected from 1-10, such as 1, 2, 3, 4, 5, 6, 7, 8, 9, or 10.

The antibody, antibody fragment, albumin, fatty acid or any other one of the half-life extending can be conjugated to PrlR-A and/or GHR-A via any suitable linker or linker region. The linker may be a disulphide bridge, such as a —S—S— bond between two cysteine (Cys) amino acid residues in each of the PrlR-A and/or GHR-A, and the pharmaceutically acceptable molecule. The linker may be a fused linker meaning that PrlR-A and/or GHR-A can be expressed in a living cell as one polypeptide or protein. The linker may be a hydrophilic linker that separates an PrlR-A and/or GHR-A and a pharmaceutically acceptable molecule with a chemical moiety, which comprises at least 5 non-hydrogen atoms where 30-50% of these are either N or O. The linker may be hydrolysable as described in U.S. Pat. No. 6,515,100, U.S. Pat. No. 7,122,189, U.S. Pat. No. 7,700,551, WO2004/089280, WO2006/138572 and WO2009/095479. Typical compounds useful as linkers in the present invention include those selected from the group having dicarboxylic acids, malemido hydrazides, PDPH, SPDP, LC-SPDP, GMBS, carboxylic acid hydrazides, and small peptides. More specific examples of compounds useful as linkers, according to the present invention, include: (a) dicarboxylic acids such as succinic acid, glutaric acid, and adipic acid; (b) maleimido hydrazides such as N-[maleimidocaproic acid]hydrazide (EMCH), N-[maleimidopropionic acid]hydrazide (MPH or BMPH), 4-[N-maleimidomethyl]cyclohexan-1-carboxylhydrazide, and N-[k-maleimidoundcanoic acid]hydrazide (KMUH), 4-(4-N-MaleimidoPhenyl)butyric acid Hydrazide (MPBH); (c) NHS-3-maleimidopropionate Succinimide ester (MPS-EDA); (d) PDPH linkers such as (3-[2-pyridyldithio]propionyl hydrazide) conjugated to sulfurhydryl reactive protein; (e) N-Succinimidyl 3-(2-pyridyldithio)-propionate (SPDP), (f) Succinimidyl 6-(3-[2-pyridyldithio]-propionamido)hexanoate (LC-SPDP), (g) N-(v-Maleimidobutyryloxy)succinimide ester (GMBS), and (h) carboxylic acid hydrazides selected from 2-5 carbon atoms. Other non-peptide linkers are also possible. For example, alkyl linkers such as —NH—(CH2)m-C(0)-, wherein m is an integer selected from 2-20, could be used. These alkyl linkers may further be substituted by any non-sterically hindering group such as lower alkyl (e.g., C1 to C6) lower acyl, halogen (e.g., Cl, Br, I, F), CN, NH2, phenyl, etc. An exemplary non-peptide linker is a PEG linker. Additional linkers useful according to the present invention are described in U.S. Pat. No. 6,660,843.

Different techniques for linking two or more molecules together, such as PrlR-A and/or GHR-A and the pharmaceutically acceptable molecule, and optionally via a multifunctional linker, such as bifunctional linker, are available in the prior art, and a suitable reference here is WO01/58493, including all relevant documents listed and cited therein.

In the present context, the term “a pharmaceutically acceptable molecule” as used herein means a molecule selected from any one of small organic molecules, peptides, oligopeptides, polypeptides, proteins, receptors, glycosylations, sugars, polymers (e.g. polyethylene glycols, PEG), nucleic acids (e.g. DNA and RNA), hormones, which when linked to PrlR-A and/or GHR-A, increases the serum half-life of the PrlR-A and/or GHR-A or variant thereof. Typically, pharmaceutically acceptable molecules are without limitation albumin, such as human albumin, recombinant albumin, or polymer, such as PEG, e.g. PEG of a molecular weight of at least 10 kDa, such as from 10 kDa to 150 kDa. Furthermore, pharmaceutically acceptable molecules may be selected from a Fc fragment of a mammalian antibody, transferrin, albumin, such as human albumin, recombinant albumin, variants of albumin, CH₃(CH₂)ₙCO—, wherein n is 8 to 22, or polymer, such as PEG, e.g. PEG of a molecular weight of at least 5 kDa, such as from 10 kDa to 150 kDa, typically 10 to 40 kDa.

In the present context, the term “in vivo plasma half-life” is used in its normal meaning, i.e., the time required for the amount of PrlR-A and/or GHR-A, in a biological system to be reduced to one half of its value by biological processes.

The term “serum half-life”, which may be used interchangeably with “plasma half-life” or “half-life” is used in its normal meaning, i.e., the time required for the amount of PrlR-A and/or GHR-A in a biological system to be reduced to one half of its concentration. Thus as used herein, the “serum half-life” means the serum half-life in vivo. Determination of serum half-life is often more simple than determining functional half-life and the magnitude of serum half-life is usually a good indication of the magnitude of functional in vivo half-life. Preferably the serum half-life is measured in a mammal, more preferably in a species of Hominidae, such as Orangutan, Chimpanzee or Gorillas, more preferably in humans. The serum half-lives mentioned in the present application are half-lives as determined in humans. An indication of the half-life or any change in half-life can also be obtained in rodents, such as mouse or rat or hamster. Furthermore half-life can be measured in larger mammals having a body weight in the same range as human beings or closer to human being body weight than rodents: preferably monkey, dog, pig, or cattle (calf).

The term “increased” as used in connection with the plasma half-life is used to indicate that the relevant half-life of the PrlR-A and/or GHR-A compound, as determined under comparable conditions. For instance the relevant half-life may be increased by at least about 25%, such as by at least about 50%, e.g., by at least about 100%, 150%, 200%, 250%, or 500%. Measurement of in vivo plasma half-life can be carried out in a number of ways as described in the literature. An increase in in-vivo plasma half-life may be quantified as a decrease in clearance or as an increase in mean residence time (MRT). The PrlR-A and/or GHR-A compound of the present invention for which the clearance is decreased to less than 70%, such as less than 50%, such as less than 20%, such as less than 10% of the clearance of the PrlR-A and/or GHR-A, as determined in a suitable assay is said to have an increased in-vivo plasma half-life. PrlR-A and/or GHR-A of the present invention for which MRT is increased to more than 130%, such as more than 150%, such as more than 200%, such as more than 500% of the MRT of PrlR-A and/or GHR-A, in a suitable assay is said to have an increased in vivo plasma half-life. Clearance and mean residence time can be assessed in standard pharmacokinetic studies using suitable test animals. It is within the capabilities of a person skilled in the art to choose a suitable test animal for a given protein. Tests in human, of course, represent the ultimate test. Suitable test animals include normal, Sprague-Dawley male rats, mice and cynomolgus monkeys. Typically the mice and rats are injected in a single subcutaneous bolus, while monkeys may be injected in a single subcutaneous bolus or in a single iv dose. The amount injected depends on the test animal. Subsequently, blood samples are taken over a period of one to ten days as appropriate (depending on the sensitivity of the assay it may be as long as 30 days) for the assessment of clearance and MRT. The blood samples are conveniently analysed by ELISA techniques or other immunological techniques.

In the present context, the term “plasma concentration” as used herein means the concentration that can be measured in circulation at any given time after injection of PrlR-A and/or GHR-A. In the present context, the term “an injection” as used herein means administration by the parenteral route such as by subcutaneous, intramuscular, intraperitoneal or intravenous injection by means of a syringe or other administration device.

The most abundant protein component in circulating blood of mammalian species is serum albumin, which is normally present at a concentration of approximately 3 to 4.5 grams per 100 millilitres of whole blood. Serum albumin is a blood protein of approximately 70,000 Dalton (Da) which has several important functions in the circulatory system. It functions as a transporter of a variety of organic molecules found in the blood, as the main transporter of various metabolites such as fatty acids and bilirubin through the blood, and, owing to its abundance, as an osmotic regulator of the circulating blood. In the present context, the term “an albumin” as used herein means albumin of mammalian origin or non-mammalian origin, such as human serum albumin that is described in Peters, T., Jr. (1996) All about Albumin: Biochemistry, Genetics and Medical, Applications pp 10, Academic Press, Inc., Orlando (ISBN 0-12-5521 10-3), or recombinant human albumin, or modified albumin, such as human albumin modified as described in WO2011051489 and WO2010092135. WO2011051489 the specification relates to variants of a parent albumin having altered plasma half-life compared with the parent albumin. The present invention also relates to fusion polypeptides and conjugates comprising said variant albumin.

WO2010092135 based on the three-dimensional structure of albumin, the inventors have designed variant polypeptides (muteins) which have one or more cysteine residues with a free thiol group (hereinafter referred to as “thio-albumin”). The variant polypeptide may be conjugated through the sulphur atom of the cysteine residue to a conjugation partner such as a bioactive compound.

WO2005054286 the specification relates to proteins comprising Interleukin 11 (IL-11) (including, but not limited to, fragments and variants thereof), which exhibit thrombopoietic or antiinflammatory properties, fused to albumin (including, but not limited to fragments or variants of albumin).

WO2004083245 describes a composition having a greater half-life than naturally produced albumin in a patient with MS, the composition comprising an albumin-like first polypeptide bound to a second polypeptide.

WO03066681 describes a composition comprising a non-albumin protein stabilised by the addition of a highly purified recombinant human serum albumin. The non-albumin protein may be Factor VIII.

In a further aspect the present invention relates to a method of preparing a long acting biologically active PrlR-A and/or GHR-A compound, such as any one of the herein disclosed conjugates of the present invention, comprising a PrlR-A and/or GHR-A polypeptide linked to a pharmaceutically acceptable molecule, the method comprising reacting a PrlR-A and/or GHR-A with a linker attached to a pharmaceutically acceptable molecule, or reacting a PrlR-A and/or GHR-A polypeptide with a linker and then attaching said linker to a pharmaceutically acceptable molecule, or reacting a linker with a pharmaceutically acceptable molecule and then reacting a PrlR-A and/or GHR-A polypeptide with the linker attached to the pharmaceutically acceptable molecule, or by expressing the PrlR-A and/or GHR-A polypeptide and the pharmaceutically acceptable molecule from a host cell.

In one embodiment the present invention relates to a long-acting modified mammalian PrlR-A and/or GHR-A, e.g. human PrlR-A and/or GHR-A linked to such as fused to albumin, or conjugated to an acylation group or PEG and provides an in vivo plasma half-life of the mammalian PrlR-A and/or GHR-A or analog thereof, or the modified PrlR-A and/or GHR-A polypeptide which is from 2 to 48 hours in a mammal. The modified long acting PrlR-A and/or GHR-A is believed to improve patient convenience and treatment outcome by reducing the frequency of PrlR-A and/or GHR-A administration.

In another embodiment, increased half-life is obtained by use of a sustained delivery system or slow release delivery. For example, liposomes are well-known drug carriers, which could be employed for delivery of polypeptides of the present invention. In this case, liposomes could be produced, which comprise a PrlR-A and/or GHR-A polypeptide of the invention. Sustained delivery systems based on the biodegradable polymers poly(lactic acid) (PLA) and poly(lactic/glycolic acid) (PLGA) are also suitable for delivery of polypeptide drugs of the present invention.

In one embodiment the composition of the invention is modified in order to increase its half-life when administered to a patient, in particular its plasma half-life. The modification may be in the form of a moiety conjugated to the composition of the invention, thus generating a moiety-conjugated composition, wherein said moiety-conjugated composition has a plasma and/or serum half-life being longer than the plasma and/or serum half-life of the non-moiety conjugated composition. In one such embodiment the moiety conjugated to the composition is one or more type of moieties selected from the group consisting of albumin and variants thereof, fatty acids, polyethylene glycol (PEG), acylation groups, antibodies and antibody fragments. The conjugation of the moiety to the polypeptide of the invention may be to any suitable amino acid residue (backbone or side chain) of the polypeptide of the invention. The moiety may also be conjugated to polypeptide of the invention by a linker. In certain embodiments said linker has a sequence selected from the group consisting of SEQ ID NO:67, 68, 69, 70 and 71.

In one embodiment the moiety conjugated to the polypeptide according to the present invention is a moiety which facilitates transport across the blood brain barrier (BBB). An example of such a cross-BBB transport facilitator is an antibody from a camelid species. Camelids such as dromedaries, camels, llamas, alpacas, vicuñas, and guanacos have single-chain antibodies capable of crossing the BBB. The person of skill in the art is aware of how to See Li et al (2012) FASEB J. (10):3969-79

Thus in one embodiment the PrlR-A and/or GHR-A polypeptide of the invention further comprises a moiety conjugated to said polypeptide, thus generating a moiety-conjugated polypeptide.

In one embodiment the moiety-conjugated composition has a plasma and/or serum half-life being longer than the plasma and/or serum half-life of the non-moiety conjugated composition.

In one embodiment the moiety facilitates transport across the blood brain barrier. This may be achieved e.g. by as a ‘moiety’ using an antibody from a camelid species such as a recombinant or native single-chain antibody from dromedaries, camels, llamas, alpacas, vicuñas, or guanacos.

In one embodiment the moiety conjugated to the PrlR-A and/or GHR-A polypeptide is one or more type(s) of moieties selected from the group consisting of albumin, fatty acids, polyethylene glycol (PEG), acylation groups, antibodies and antibody fragments.

In one embodiment the PrlR-A and/or GHR-A polypeptide are linked to the moiety by a linker. In one embodiment the moiety is conjugated to the PrlR-A and/or GHR-A polypeptide.

In one aspect the invention concerns a polypeptide selected from the group consisting of 7, 8, 16, 17, 18, 19, 20, 21, 22, 23, 24, 25, 26, 27, 28, 29, 30, 31, 32, 32 and 33 or a fragment or variant thereof disclosed herein, as well as methods of manufacturing said polypeptide or said variant or fragment.

The composition of the present invention which comprises a PrlR-A and/or a GHR-A can also be administered as a ‘pro-drug’ in the form of a nucleotide, e.g. as an expression vector comprising the nucleotide encoding the PrlR-A and/or GHR-A polypeptide(s).

In one embodiment, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector may be an expression vector in which the DNA sequence encoding the peptide of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the peptide.

Typically the expression is directed by a promoter. Thus in one embodiment the invention concerns a vector which further comprises a promoter operably linked to the nucleic acid sequence encoding the PrlR-A and/or GHR-A polypeptide.

The promoter may be any DNA sequence which shows transcriptional activity in the host cell of choice and may be derived from genes encoding proteins either homologous or heterologous to the host cell.

The promoter may be any suitable promoter e.g. a promoter is selected from the group consisting of: CMV, human UbiC, RSV, Tet-regulatable promoter, Mo-MLV-LTR, Mx1, EF-1alpha, PDGF beta and CaMK II. Examples of suitable promoters for use in yeast host cells include promoters from yeast glycolytic genes (Hitzeman et al., J. Biol. Chem. 255, 12073-12080 (1980); Alber and Kawasaki, J. Mol. Appl. Gen. 1, 419-434 (1982)) or alcohol dehydrogenase genes (Young et al., in Genetic Engineering of Microorganisms for Chemicals (Hollaender et al, eds.), Plenum Press, New York, 1982), or the TPM (U.S. Pat. No. 4,599,311) or ADH2-4c (Russell et al., Nature 304, 652-654 (1983)) promoters.

Examples of suitable promoters for use in filamentous fungus host cells are, for instance, the ADH3 promoter (McKnight et al., The EMBO J. 4, 2093-2099 (1985)) or the tpiA promoter. Examples of other useful promoters are those derived from the gene encoding A. oryzae TAKA amylase, Rhizomucor miehei aspartic proteinase, A. niger neutral [alpha]-amylase, A. niger acid stable [alpha]-amylase, A. nigeror A. awamori glucoamylase (gluA), Rhizo-mucor miehei lipase, A. oryzae alkaline protease, A. oryzae triose phosphate isomerase or A. nidulans acetamidase. In one embodiment, the promoter of a vector according to the invention is selected from the TAKA-amylase or the gluA promoters. Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gen, or the Bacillus pumilus xylosidase gene, or by the phage Lambda PR or PL promoters or the E. coli lac, trp or tac promoters. The DNA sequence encoding the peptide of the invention may also, if necessary, be operably connected to a suitable terminator, such as the human growth hormone terminator (Palmiter et al., op. cit.) or (for fungal hosts) the TPM (Alber and Kawasaki, op. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The vector or vectors comprising the nucleotide sequences which upon expression encodes PrlR-A and/or GHR-A polypeptide(s) are selected by the person of skill in the art based on the target tissue and indication to be treated. The vector may e.g. be selected from the group consisting of vectors derived from the Retroviridae family including lentivirus, HIV, SIV, FIV, EAIV, CIV, or from the group consisting of adeno associated virus, adenovirus, alphavirus, baculovirus, HSV, coronavirus, Bovine papilloma virus, Mo-MLV.

In some embodiments it may be advantageous to administer to the individual a cell, e.g. a cell encapsulated in a semipermeable biocompatible capsule. The cell comprising a nucleotide encoding a PrlR-A and/or GHR-A polypeptide may be selected from the group consisting of Saccharomyces cerevisiae, E. coli, Aspergillus and insect cells such as Sf9 insect cells or any other cell suitable for the intended purpose.

The host cell may thus also be selected from the group consisting of mammalian cells selected from the group consisting of human, feline, porcine, simian, canine, murine and rat cells.

In one embodiment the host cell is selected from the group consisting of CHO, CHO-K1, HEI193T, HEK293, COS, HiB5, RN33b and BHK cells.

Indications

The need for a test system to determine GH/Prl sensitivity according to the present invention is linked to certain medical conditions as indicated below and these indications are also conditions where compounds capable of blocking GH/Prl receptors are useful as medicaments.

The expression pattern outlined in table 1 and 2 above forms the basis for diagnosing the individual from which the sample has been obtained, with one or more of the following indications, and to select the appropriate method of treatment of the specific indication.

Class C: C18 colon cancer, C22 Liver cancer, C50 Breast cancer, C55 Endometrial cancer, C56 Ovary cancer, C61 Prostate cancer,

C75 Parathyroid cancer

Class D: D24 Benign breast tumour, D25 Leiomyoma, D41 renal angiomyolipoma

Class E: E22 Acromegaly, E22.1 Hyperprolactinemia, E65-68 Obesity

Class J: J84 Lymphangioleiomyomatosis (LAM)

Class L: L93 Lupus erythematosus

Class N: N40 Benign prostate tumour

Class O: O90 Peripartum cardiomyopathy

The list of indications above listed in accordance with the ICD-10 classification system comprises benign and malign tumours, inflammation and endocrine states associated with the biomarkers of the present invention (GHR, PrlR, SOCS2, TCS2). While some of these indications have different aetiology and symptoms, the nexus between is that they all represent disturbed growth of particular cell types and, in common, respond to compositions interfering with growth and metabolic signals. GH and Prl activate growth promoting signals e.g. the STAT system and these hormone also change intracellular metabolism. The latter is related to a system sensing intracellular energy levels, the so called AMPK system.

Intracellular growth signals leading to proliferation include a variety of kinases and these are frequently discussed as cancer drug targets. Results further show cancer preventive effects of the anti-diabetic drug Metformin and the target for this drug is likely to be changes in cell energy (ATP/ADP) ratio—therefore interference with cell metabolism prevent cancer.

Blocking GHR and/or PrlR, results in reduced growth related signalling and altered cell metabolism (in analogy with the Metformin effect), thus impeding tumour growth. Current cancer treatments used in the clinic, target relatively broad cell functions (c.f. radiation and DNA replication). The present invention also targets broad cell functions namely growth signals and metabolism but it is here important to ensure that a tumour has the ability to respond to GH/Prl and that there are recompositions that de facto block GH and Prl actions. Below is a description of indications that respond to GHR and/or PrlR antagonists. Due to tumour heterogeneity, not all tumours will respond and it is thus a need for mapping of the expression pattern in line with the present invention.

The pathological mechanisms of the present invention demonstrate a relationship between the indications of the invention, thus evidencing the nexus between the indication and the method of treatment of the present invention. Antagonizing GHR/PrlR will principally affect both growth related and metabolic signals and this will broadly influence tumour cells to change growth and metabolism in a manner that is unfavourable for tumour growth. Several intracellular proteins or secreted proteins are activated by GH/Prl and the literature is extensive on the potential value of drug interference with individual components of GH/Prl activated signals (e.g. the STATs or the IGF-I system). According to the present invention it is advantageous to block at the receptor level rather than at the level of downstream effectors molecules. A prerequisite is of course that GHR and/or PrlR exists on the target tissues and that the sensitivity for GH and/or Prl is high. The indications described herein are related to either GH or Prl. In reality the distinction between GH or Prl is less distinct and even overlapping. For this reason the measurement of the sensitivity to GH and/or Prl is a key factor as put forward in this patent application.

The literature on the indications listed above is very extensive and it is also clear that the hormones GH and Prl have been frequently mentioned in conjunction with these disorders. The mentioning of elevated hormone levels or the presence of GH and/or Prl receptors in a particular form of tumour is in many cased followed by suggestions that reduction of elevated hormone levels or blocking receptors may affect tumour growth. Such statements are of course adequate to motivate an investigation but in the absence of any innovative steps or clinical/experimental tests it is only to be regarded as suggestions. In the present invention we define a test system that will distinguish between non-responding and responding tumours, we also provide the use of high affinity PrlR antagonists and the realization that combined treatment with PrlR and GHR can be advantageous in some situations.

C18 Colon Cancer

Colorectal cancer, commonly known as colon cancer or bowel cancer, is a cancer from uncontrolled cell growth in the colon or rectum (parts of the large intestine), or in the appendix. Genetic analysis shows that colon and rectal tumours are essentially genetically the same cancer. Symptoms of colorectal cancer typically include rectal bleeding and anemia which are sometimes associated with weight loss and changes in bowel habits.

Most colorectal cancer occurs due to lifestyle and increasing age with only a minority of cases associated with underlying genetic disorders. It typically starts in the lining of the bowel and if left untreated, can grow into the muscle layers underneath, and then through the bowel wall. Screening is effective at decreasing the chance of dying from colorectal cancer and is recommended starting at the age of 50 and continuing until a person is 75 years old. Localized bowel cancer is usually diagnosed through sigmoidoscopy or colonoscopy. Cancers that are confined within the wall of the colon are often curable with surgery while cancer that has spread widely around the body is usually not curable and management then focuses on extending the person's life via chemotherapy and improving quality of life. Colorectal cancer is the third most commonly diagnosed cancer in the world, but it is more common in developed countries. Around 60% of cases were diagnosed in the developed world. It is estimated that worldwide, in 2008. PrlR and GHR are present in colon cancer and reports suggest that elevated Prl is a marker for colon cancer. Tests for GH/Prl sensitivity according to the invention can be carried out using sections of colon cancer tumours and to determine control levels, adjacent tissue or a section of normal colon can be used.

C22 Liver Cancer

Hepatocellular liver cancer (HCL) is the third most common form of cancer. The liver is furthermore a common site for cancer metastasises. GHR and PrlR are present in the liver and activation of these receptors lead to changes of growth and metabolism of the liver. In addition the liver is the main site for IGF-I production that is controlled by GH. Persistently high GH levels are believed to cause liver cancer. Tests for GH/Prl sensitivity according to the invention can be carried out using biopsy material from liver cancer and to determine control levels, adjacent normal tissue or a section of normal liver can be used.

C50 Breast Cancer

Breast cancer exists in different forms and has genetic and environmental causes. Certain signal transduction pathways e.g. the PI3K/AKT and RAS/MEK/ERK pathway are changed because of mutations in signalling components. Breast cancer cells may or may not have three important receptors: estrogen receptor (ER), progesterone receptor (PR), and HER2. Determination of these receptor levels is important because it directs how the tumour should be treated. Prl acts on normal breast to induce growth, differentiation and milk production. Since Prl is produced in cancer cells the use of PrlR antagonists can be a future treatment modality. Tests for GH/Prl sensitivity according to the invention can be carried out using biopsy material from breast cancer and to determine control levels, adjacent normal tissue or material from normal breast can be used.

C55 Endometrial Cancer

Endometrial cancer is the most common gynaecological cancer and is most commonly treated with hysterectomy. Risk factors include e.g. infertility, estrogen elevation and obesity. Elevation of prolactin is a frequent finding as are increases of PrlR in this form of cancer. Tests for GH/Prl sensitivity according to the invention can be carried out using material from endometrial cancer and to determine control levels material from normal endometrium can be used.

C56 Ovary Cancer

Cancer in the ovary can appear without major symptoms and may therefore be un-diagnosed for some time. The tumour is related to the hormonal status and also to heredity of certain predisposing genes. Prl increases the survival and migration of ovarian cancer cells and the PrlR is increased in the tumours. This situation indicates that a PrlR antagonist may have a future clinical value. Tests for GH/Prl sensitivity according to the invention can be carried out using material from ovary cancer and to determine control levels to determine normal levels, material from normal endometrium can be used.

C61 Prostate Cancer

Prostate cancer is a common cancer diagnose in elderly men. Yet unclear genetic components and environmental factors are behind prostate cancer. Once diagnosed, it can be categorized in different forms (Gleason score). Prostate cancer cells are dependent on androgen for growth and for this reason, blockers of androgen actions are commonly used for treatment. Unfortunately some tumours stop responding to treatment and androgen resistance prostate cancer is still without any cure. Prl/GH stimulates growth of prostate cells and takes part in the actions of androgens and Prl is also produced by prostate cancer cells. Tests for GH/Prl sensitivity according to the invention can be carried out using material from prostate cancer and to determine control levels adjacent normal tissue or material from normal prostate can be used.

C75 Parathyroid Cancer

Parathyroid carcinoma is uncommon whereas adenomas are more common. The presence of PrlR and the hormonal control of calcium suggest that PrlR antagonist would influence parathyroid tumours. Tests for GH/Prl sensitivity according to the invention can be carried out using material obtained at surgery and to determine control levels adjacent normal tissue or material from normal parathyroid glands can be used.

D24 Benign Breast Tumour

Benign breast tumours, fibroids, are hormone dependent non-malignant breast tumours. Although different from breast cancer these fibroids can present a clinical problem and can hide malignancies. Prl is one hormone that has been connected to this situation. Tests for GH/Prl sensitivity according to the invention can be carried out using biopsy material and to determine control levels material from normal breast can be used.

D25 Leiomyoma

Leiomyomas are smooth muscles tumours. Benign leoimyomas are common in the uterus and a sub-form, lipoleiomyoma has been connected to ovarian and other pathologies. The malignant form, leiomyosarcoma is uncommon and can have very different clinical courses. Prl and PrlR can be detected in leiomyomas at elevated levels. Tests for GH/Prl sensitivity according to the invention can be carried out using biopsy material and to determine control levels adjacent normal tissue or material from normal uteri can be used.

D41 Renal Angiomyolipoma

Angiomyolipomas are the most common benign tumour of the kidney. A common genetic finding in these tumours is reduction of TSC1/TSC2. Tests for GH/Prl sensitivity according to the invention can be carried out using biopsy material and to determine control levels adjacent normal tissue or material from normal kidney can be used.

E22 Acromegaly

Acromegaly is caused by over production of GH from pituitary adenomas. Somatostatin (or analogues) is used for medical treatment with the aim to reduce GH secretion. In cases when somatostatin is not working, GHR antagonists are used. It is possible that pituitary adenomas also secrete Prl and this can motivate a combined treatment with GHR/PrlR antagonists. This diagnosis is not suitable for tests according to the invention and instead treatment using GH receptor antagonists is based on the failure of observing a treatment response of drugs acting on the hypothalamo-pituitary level.

E22.1 Hyperprolactinemia

One effect of hyperprolactinemia is infertility and long term elevations of serum Prl is a risk factor for tumour developments. The source of hyperprolactinemia is a pituitary adenoma. Most cases can be treated with compositions blocking the dopamine receptor but there are reports that around 10% fail to respond to this treatment. In such cases the remaining logical treatment would be to block the PrlR. Dopamine resistant hyperprolactinemia is diagnosed by a failure of dopamin receptor agonists to reduce serum prolactin and consequently the test system described in the present invention cannot be applied in this case.

E65-68 Obesity

Obesity has connection to hormones. Prl increases fat deposition in adipose tissue and the absence of PrlR reduces the effect of high fat feeding in animals. An antagonist of PrlR will mimic these effects. Biopsies of adipose tissue can be used to analyse GH/Prl sensitivity to ensure a treatment response. As control one will need a panel or normal fat.

J84 Lymphangioleiomyomatosis

Lymphangioleiomyomatosis (LAM) affects women in child bearing ages. LAM lesions are smooth muscle containing, benign tumours found in the lung. LAM is caused by a failure of TSC2/TSC1 to reduce mTOR and the resulting overactive mTOR activates protein synthesis and changes the ATP/ADP ratio. LAM progression can be affected by drugs blocking mTOR. In accordance with the present invention LAM is a Prl hypersensitive state and blocking the PrlR will reduce growth of LAM lesions. Tests for GH/Prl sensitivity according to the invention can be carried out using biopsy material and adjacent normal tissue can be used to determine control levels.

L93 Lupus Erythematosus

Lupus erythematosus (SLE) is a condition where the immune system becomes hyper active and attacks normal tissue. The disease manifestation is many and a changed B-cell function is an important component of the disease. The predominance of women in auto-immune disorders has led to investigations of endocrine components in SLE. Animal experiments suggest that excess Prl can aggravate SLE. It may therefore be the case that a high affinity PrlR antagonist can affect SLE. Test for Prl/GH sensitivity in immune cells can in principle be carried out using the present invention.

N40 Benign Prostate Tumour

Benign prostatic hyperplasia (BPH) is very common. The prostate gland is highly responsive to hormones including androgen, estrogen and prolactin. Animal experiment show that elevation of Prl leads to BPH and subsequently that the blocking PrlR will affect this condition. Tests for GH/Prl sensitivity according to the invention can be carried out using material from BPH and control levels can be determined in adjacent normal tissue.

O90 Peripartum Cardiomyopathy

Peripartum cardiomyopathy (PPCM) is an uncommon and life threatening condition that affect women in the last trimester of pregnancy of the first month post-partum. Because of elevated levels of Prl in this condition one has tried treatment with Prl reducing drugs (dopamine receptor agonist) with promising results. This indicates that overstimulation of PrlR has severe consequences in this condition and that blocking PrlR is an alternative to reducing serum Prl. Changes of GH/Prl sensitivity in PPCM cannot be determined as a clinical test instead treatment with PrlR-A will be aligned to other attempts to reduce actions of Prl on the heart.

Subgroups of Disorders Characterized by a Prl/GH Dependence

There are certain conditions suitable to be treated according to the present invention where material cannot be obtained or analysed using the test kit as described above. These conditions are summarized below.

Hyperprolactinemia (E22.1) which does not respond to conventional treatment by dopamine receptor agonist can be treated by a PrlR-A according to the present invention. Conditions where Prl is over-secreted by the pituitary gland i.e. hyperprolactinemia may not always respond to conventional dopamine agonist therapy. Such conventional treatment of hyperprolactinemia is based on activation of dopamine receptors, but in some cases dopamine agonists fail and a remaining option is to use Prl receptor antagonists instead. Interference with the dopamine system may occur in the treatment of psychiatric disorders, as clinically used dopamine antagonists can cause unwanted elevation of Prl. Therefore, drug induced hyperprolactinemia is a potential use for Prl receptor antagonists, as treatment of a patient with both dopamine antagonists and agonists is not an option.

SLE (L93) where the subclassification is Prl dependency can be treated with PrlR-A according to the present invention.

Acromegaly (E22) where mixed GH and Prl production does not respond to drugs acting at the hypothalamo-pituitary level can be treated with GHR-A/PrlR-A combination according to the present invention.

Obesity (E65) where the subclassification is Prl depencency can be treated with PrlR-A according to the present invention.

Peripartum cardiomyopathy (O90) can be treated with PrlR-A according to the present invention.

Psychotic disorders (F20-F29) wherein the subclassification is side effects of dopamin antagonist treatment can be treated with PrlR-A according to the present invention.

A further category of medical indications suitable for treatment with the high affinity Prl receptor antagonists according to the invention are those characterized by overproduction of Prl in the vicinity of the tumours. Such overexpression of Prl in individual tumours can be detected using different technologies based on detection of the substance as such, e.g. by Prl specific antibodies with high affinity for the target with Western blots, ELISA and immunohistochemistry, just to mention a few techniques, or some Prl related compound, e.g. Prl mRNA, e.g. by PCR technique. Measurement of Prl in so called tissue arrays can be used to compare levels of Prl in different tumours. With this technique overproduction of Prl has been detected in certain forms of prostate cancer (ductal prostate cancer), in myomas (notably leiomyomas) and in endometrical cancer.

Pharmaceutical Composition

The composition of the present invention comprising PrlR-A and/or GHR-A, can be formulated as a pharmaceutical composition.

In the present context, the term “a pharmaceutical composition” as used herein typically means a composition containing PrlR-A and/or GHR-A and/or a PrlR-A and/or GHR-A variant of the present invention, and optionally one or more pharmaceutically acceptable vehicle, carrier or excipient, and may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications. Typically, the pharmaceutical compositions of the present invention may be formulated for parenteral administration e.g., by i.v. or subcutaneous injection, and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory compositions such as preserving, wetting, emulsifying or suspending, stabilizing and/or dispersing compositions. Alternatively, the active ingredient may be in powder form, obtained by aseptic isolation of sterile solid or by lyophilisation from solution for constitution before use with a suitable vehicle, e.g., sterile, pyrogen-free water. Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. The parenteral formulations typically will contain from about 0.0001 to about 25%, such as from about 0.5 to about 25%, by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 0.000001 to about 15% by weight, such as from about 0.000001 to about 5% by weight or from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use.

The main route of drug delivery according to this invention is however parenteral in order to introduce the composition into the blood stream to ultimately target the relevant tissue.

The composition may also be administered to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth.

In a preferred embodiment the composition of the invention is administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The compounds may also be administered by inhalation, which is by intranasal and oral inhalation administration. Appropriate dosage forms for such administration, such as an aerosol formulation or a metered dose inhaler, may be prepared by conventional techniques.

In one embodiment the pharmaceutical composition according to the present invention is formulated for parenteral administration such as by injection.

In a further embodiment the pharmaceutical composition according to the present invention is formulated for intravenous, intramuscular, intraspinal, intraperitoneal, subcutaneous, a bolus or a continuous administration.

The rate and frequency of the administration may be determined by the physician from a case to case basis. In one embodiment the administration occurs at intervals of 30 minutes to 24 hours, such as at intervals of 1 to 6 hours.

The duration of the treatment may vary depending on severity of the condition. In one embodiment the duration of the treatment is from 6 to 72 hours. In chronic cases the duration of the treatment may be lifelong.

The dosage can be determined by the physician in charge based on the characteristics of the patient and the means and mode of administration. In one embodiment of the present invention, the dosage of the active ingredient of the pharmaceutical composition as defined herein above, is between 10 μg to 500 mg per kg body mass, such as between 20 μg and 400 mg, e.g. between 30 μg and 300 mg, such as between 40 μg and 200 mg, e.g. between 50 μg and 100 mg, such as between 60 μg and 90 μg, e.g. between 70 μg and 80 μg.

The dosage may be administered as a bolus administration or as a continuous administration. In relation to bolus administration the pharmaceutical composition may be administered at intervals of 30 minutes to 24 hours, such as at intervals of 1 to 6 hours. When the administration is continuous it is administered over an interval of time that normally is from 6 hours to 7 days. However, normally the dosage will be administered as a bolus 1-3 times per day.

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier therefore.

In one embodiment the pharmaceutical composition as defined herein above comprises a pharmaceutically acceptable carrier.

The compositions of the present invention may be formulated into a wide variety dosage forms, suitable for the various administration forms discussed above.

The pharmaceutical compositions and dosage forms may comprise the compositions of the invention or its pharmaceutically acceptable salt or a crystal form thereof as the active component.

Furthermore, the pharmaceutical compositions may comprises pharmaceutically acceptable carriers that can be either solid or liquid.

Solid form preparations are normally provided for oral or enteral administration, such as powders, tablets, pills, capsules, cachets, suppositories, and dispersible granules. A solid carrier can be one or more substances which may also act as diluents, flavoring compositions, solubilizers, lubricants, suspending compositions, binders, preservatives, wetting compositions, tablet disintegrating compositions, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. Powders and tablets preferably contain from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal composition and/or any other suitable preservative, and optionally including a surface active composition. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal compositions suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing compositions, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying compositions such as lecithin, sorbitan monooleate, or acacia. Aqueous solutions can be prepared by dissolving the active component in water and adding suitable colorants, flavours, stabilizing and thickening compositions. Aqueous suspensions can be prepared by dispersing the finely divided active component in water with viscous material, such as natural or synthetic gums, resins, methylcellulose, sodium carboxymethylcellulose, and other well known suspending compositions. Solid form preparations include solutions, suspensions, and emulsions, and may contain, in addition to the active component, colorants, flavours, stabilizers, buffers, artificial and natural sweeteners, dispersants, thickeners, solubilizing compositions, and the like.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory compositions such as preserving, wetting, emulsifying or suspending, stabilizing and/or dispersing compositions. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The compounds of the invention can also be delivered topically for transdermal or transmucosal administration. Regions for topical administration include the skin surface and also mucous membrane tissues of the vagina, rectum, nose, mouth, and throat. Compositions for topical administration via the skin and mucous membranes should not give rise to signs of irritation, such as swelling or redness. Transdermal administration typically involves the delivery of a pharmaceutical composition for percutaneous passage of the drug into the systemic circulation of the patient. The skin sites include anatomic regions for transdermally administering the drug and include the forearm, abdomen, chest, back, buttock, mastoidal area, and the like.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, such as a sublingual tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling compositions. Lotions may be formulated with an aqueous or oily base and will in general also containing one or more emulsifying compositions, stabilizing compositions, dispersing compositions, suspending compositions, thickening compositions, or colouring compositions. Formulations suitable for topical administration in the mouth include lozenges comprising active compositions in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active composition such as an anionic, cationic or non-ionic surfactant such as a sorbitan ester or a polyoxyethylene derivative thereof. Suspending compositions such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include a composition to hasten drying and to cool the skin, such as an alcohol or acetone, and/or a moisturizer such as glycerol or an oil such as castor oil or arachis oil.

Transdermal delivery may be accomplished by exposing a source of the complex to a patient's skin for an extended period of time. Transdermal patches have the added advantage of providing controlled delivery of a pharmaceutical composition-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical composition-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

For example, a simple adhesive patch can be prepared from a backing material and an acrylate adhesive. The pharmaceutical composition-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Foam matrix patches are similar in design and components to the liquid reservoir system, except that the gelled pharmaceutical composition-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

The compounds of the present invention may also be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example with a dropper, pipette or spray. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The pH of the pharmaceutical composition may be any pH suitable for physiological purposes such as between pH 4 and pH 10, preferably between pH 4 and pH 9, preferably between 5 and 8, more preferably around pH 7.

According to a further aspect of the invention, the antagonist is administered by injection or by oral delivery, optionally in combination with a pharmaceutically acceptable vehicle.

According to one aspect of the invention a method for treatment and/or prevention of a prolactin-related disease in a patient is provided, the method comprises administration to the patient a pharmaceutically acceptable and effective dose of one or both of prolactin receptor antagonists as defined herein.

In one embodiment the composition according to the invention is formulated for oral administration.

In another embodiment the composition according to the present invention is formulated for parenteral administration, such as by injection.

In one embodiment the composition is route of administration is intravenous, intramuscular, intraspinal, intraperitoneal, subcutaneous, a bolus or a continuous administration.

The composition according to the invention may be administered at intervals of 30 minutes to 24 hours, such as at intervals of 1 to 6 hours, such as three times a day.

Dependent on the dosage and individual requirement the composition may be administered in various time intervals, such as wherein the duration of the treatment is from 6 to 72 hours, or wherein the duration of the treatment is from 24 hours to 7 days, or wherein the duration of the treatment is from 4 days to 150 days. In one embodiment the duration of the treatment is lifelong.

The dosage of the active ingredient, i.e. PrlR-A and/or GHR-A is determined by the medically trained personnel on a case to case basis. Typically the dosage of the active ingredient is between 10 μg to 500 mg per kg body mass, such as from 50 μg to 250 mg per kg body mass.

Kit

The present invention concerns medical conditions in which the test kit according to the invention can be used for determining altered GH/Prl sensitivity. If the result of testing is that a tumour has an increased sensitivity to GH/Prl, the question to treat the tumour with compounds that reduce GH/Prl sensitivity will be raised. A GH receptor antagonist is in clinical use. The present invention in one embodiment concern use of compounds capable of blocking the Prl receptor.

In one embodiment the invention concerns a kit comprising the pharmaceutical composition as defined herein above, and instructions for use, wherein the instructions comprises table 1 or a subset of table 1, such as table 2.

Computer Implemented Methods

The methods of the present invention defined herein above are in one aspect adapted as a computer implemented method to facilitate the diagnostic steps and increase speed of treatment.

Thus in one aspect the present invention concerns a computer implemented method for selecting treatment of a prolactin-associated disorder, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) selecting a treatment regime based on table 1.

In another aspect the invention concerns a computer implemented method for diagnosing a prolactin-associated disorder, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue, wherein an expression pattern equal to the expression patterns 3, 4, 5, 6, 9, 12, 13, 14, 15, 18, 21, 22, 23, 24, 27, 30, 31, 32, 33, 36, 39, 41, 42, 45, 57, 58, 59, 60, 63, 66, 67, 68, 69, 72, 76, 77, 78 or 81 of table 1 is indicative of a prolactin associated disorder.

In one embodiment the computer implemented method defined herein above further comprises the features of the composition defined above.

In one aspect the invention concerns a computer program product having a computer readable medium, said computer program product suitable for selecting a treatment of a prolactin associated disorder in a subject based on expression patterns of the polypeptides growth hormone receptor (GHR), prolactin receptor (PrlR), suppressor of cytokine signalling 2 (SOCS2) and tuberous sclerosis complex 2 (TSC2) in tumour tissue in a sample obtained from said subject, said computer program product comprising means for carrying out all the steps of the methods defined herein above.

### EXAMPLES

Different experimental series were carried out to demonstrate the present invention:

i) use of Prl receptor antagonists to treat hyperprolactinemia and to demonstrate activity in cultured cells,
ii) hypersensitivity of Prl/GH caused by perturbations of TSC and SOCS2.
iii) an outline on the use of combined measurements of GH/Prl receptors, SOCS2 and TSC2 in clinical tumour samples.

These in vitro models are useful for demonstrating a strong indication of the in vivo potential of the Prl receptor antagonists. Prl receptor antagonists have not been tested in human subjects at this stage, but the above experiments have been designed to bear a direct relation to the human situation. In the first case a model of human hyper prolactinemia is created and this corresponds to Prl receptor antagonist treatment. The second case, a disease related to human gene changes levels of human Prl receptors. Based on the generally accepted concept that increased levels of receptors reflects increased sensitivity, one can draw the conclusion from this set of experiments that a particular human condition should respond to antagonist treatment.

### Example 1

### Prl Receptor Antagonist has an Effect In Vivo

Prl receptor antagonists (SEQ ID NO: 24) were used to circumvent consequences of hyperprolactinemia. A group of rats were infused with human Prl (5 microgram/h) using osmotic minipumps placed subcutaneously on the left flanking area on the back of each animal. The infusion of the Prl lasted for one week. One animal group received human Prl plus the Prl receptor antagonist. The high affinity Prl receptor antagonist (I) was separately infused using osmotic minipumps at a concentration of 5 microgram/h. This system mimics hyperprolactinemia because high levels of Prl are detectable in serum. The effect of hyperprolactinemia was subsequently studied using two different read-outs: changes in sex-steroids and gonadothropin follicle-stimulating hormone (FSH), luteinizing hormone (LH) and effects on liver gene expression. It was found that Prl elevation reduced levels of sex-steroids and LH/FSH. In terms of liver action, Prl also changed levels of several mRNA species notably expression of the vitamin A biogenic enzyme retinol dehydrogenase. All of the reported effects were blocked by simultaneous co-infusion of the antagonist (SEQ ID NO: 24). See table 3 below.

TABLE 3

| Animal group | Test | LH | FSH | Prl dependent genes |
|---|---|---|---|---|
| Control | — | — | — | — |
| Prl | ↓ | ↓ | ↓ | ↑ |
| Prl + antag. | ↑ | ↑ | ↑ | ↓ |

Male rats were divided into three groups each consting of 5 animals. Two groups recieved infusion of human Prl using osmotic minipums (5 ug/h for two weeks). One group was infused in parallell with osmotic minipumps containing a Prl receptor antagonist (5 ug/h for two weeks). At the end of the experiment serum was collected for analysis of leslves of testosterone (Test), LH and FSH. Livers were used to prepare RNA for measurement of gene expression. The tabel list one gene procuct, retinol dehydrogenase mRNA measured by PCR. Results are expressed as arrows indication direction of change compared or towards control untreated

### Example 2

### TCS2 Regulates Prl Receptor Levels and Prl Sensitivity

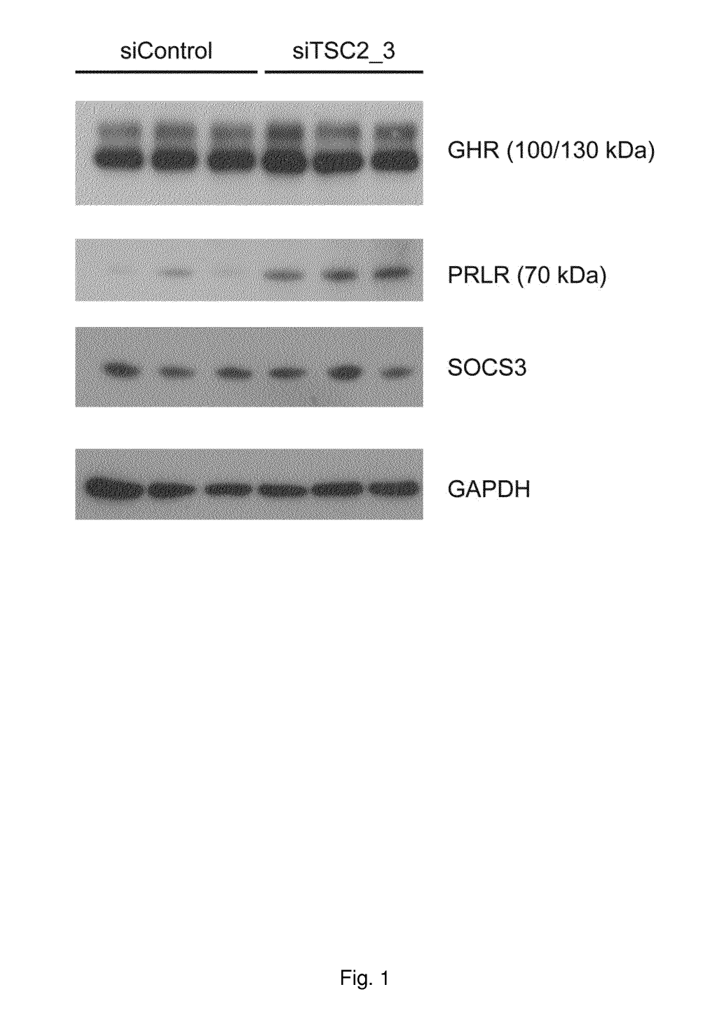

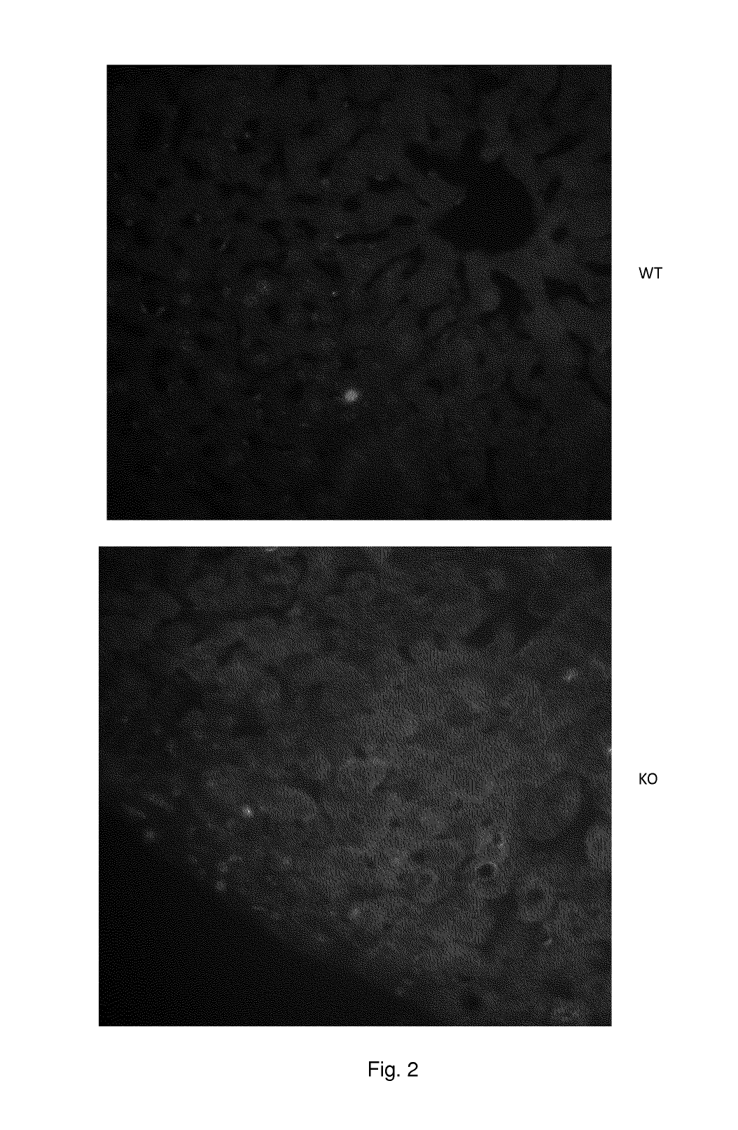

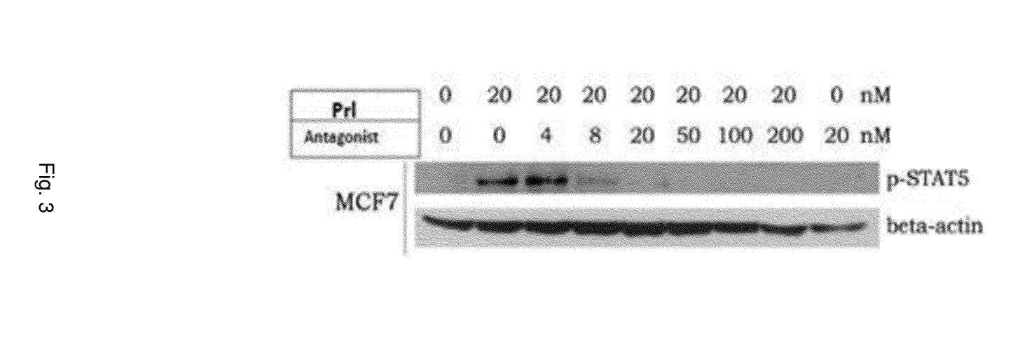

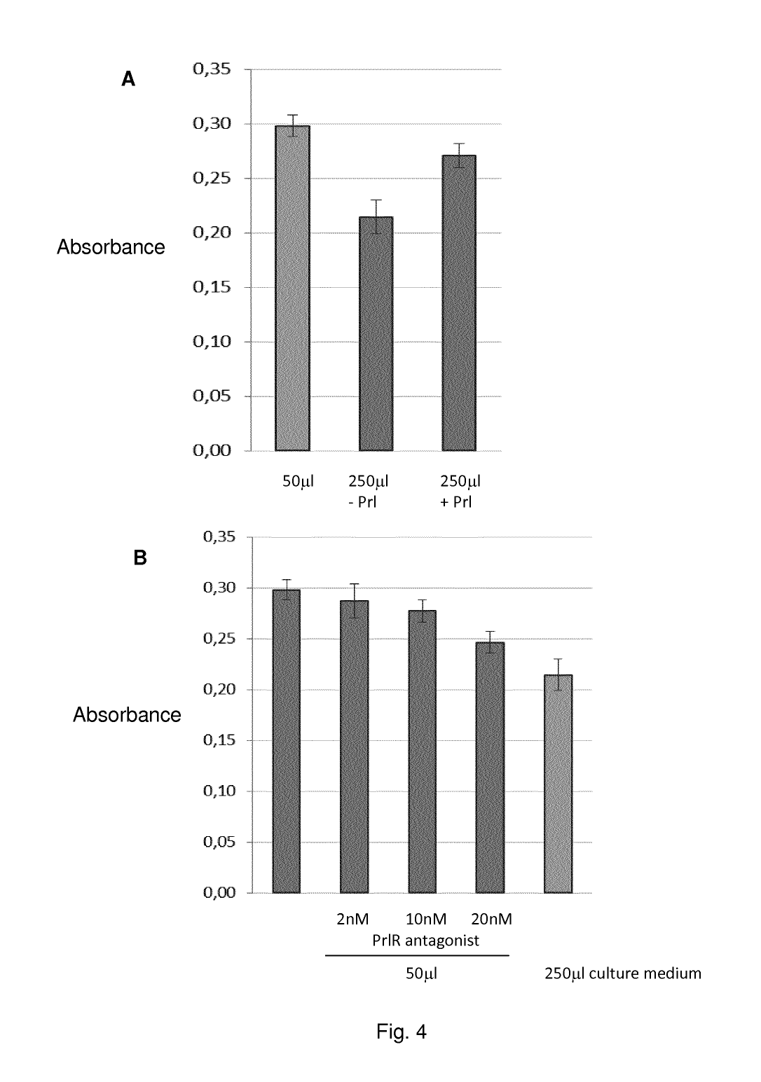

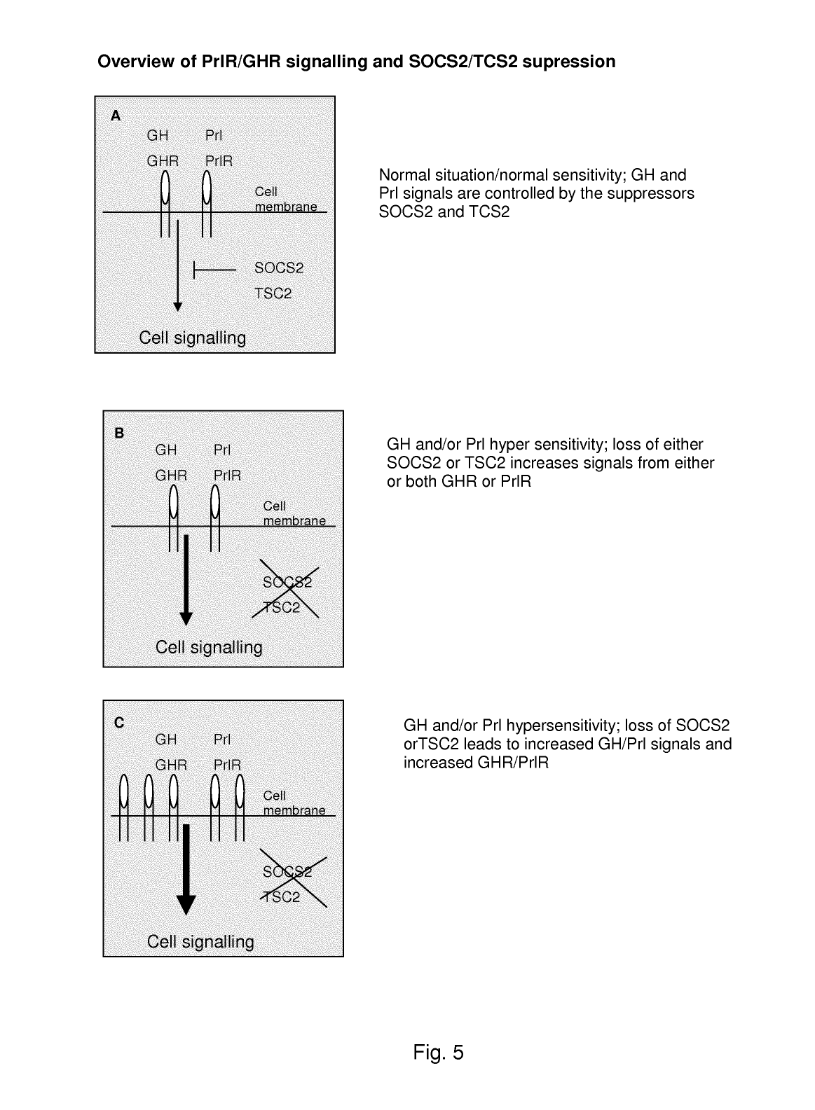

Cultured cells were used to reduce the amount of TCS2 and this was followed by measurements of the amount of Prl receptors using Western blots. FIG. 1 shows the effect of reducing TSCS2 by siRNA in myo-fibroblasts derived from Eker rats. These cells have a genetic deletion of one TCS2 allele. FIG. 1 shows that Prl receptors were increased by TCS2 siRNA. One method to demonstrate that increased Prl receptor levels lead to an increased Prl sensitivity is to add Prl (e.g. 10 nM) to cells with or without reduction of TCS2 and analyse effects on STAT activation (phosphorylation of STAT5/STA3). In such experiments we have demonstrated an increased Prl sensitivity in cells deficient of TCS2.

### Example 3

### SOCS2 Regulates Prl Receptor Levels

Mice where the SOCS2 gene was ablated were used to analyse levels of Prl receptors in liver and muscle. The experimental procedure was to prepare protein extract to be separated on poly-acryl amid gels. Proteins on the gel were subsequently transferred onto PVDF membranes that were used for anti-body detection of Prl receptors. The results show that the levels of Prl receptors were increased in these tissues and taken together with TSC2 results (cf. example 2), we conclude that two types of intracellular proteins regulate the levels of Prl receptors (FIG. 2).

### Example 4

### In Vitro Activity of the High Affinity Prl Receptor Antagonist

MCF7 breast cancer cells were stimulated with human prolactin and this leads to an activation (phosphorylation) of STAT5 as measured using Western blots. Pre-incubation of cells with the Prl receptor antagonist (SEQ ID NO: 24) blocked this effect (FIG. 3).

### Example 5

### Effect of Prl Receptor Antagonists on Cultured Breast Cancer Cells

Breast cancer cells can produce Prl and for this reason we cultured cells in a small volume of serum free medium. Serum free media eventually causes apoptosis but we found that small volume of media led to an improved cell survival which indicates that cells themselves produce survival factors. Addition of the Prl receptor antagonist (SEQ ID NO: 24) decreased survival which indicates that paracrine/autocrine Prl production is a factor of relevance for cell survival and that blocking Prl receptors reduces the survival of cancer cells (FIG. 4).

### Example 6

### Interpretation of Tissue Sections Tested for GH Receptor, Prl Receptor, SOCS2 and TSC2 in Terms of Changed GH and/or Prl Sensitivity

Tissue section from different forms of prostate cancer were stained with antibodies to detect GH receptor, Prl receptor, SOCS2 and TSC2 using immunoperoxidase techniques. The principal changes in different tumour types are indicated (Table 4).

TABLE 4
Measurement of GH receptors, prolactin receptors, SOCS2 and TSC2
in prostate cancer samples. Immunohistochemistry on tissue sections
was used to detect the above mentioned proteins in several types
of prostate cancer (stage determination according to Gleason).
The principal reactivity in such experiments can be:

| | | | A | B | C |
|---|---|---|---|---|---|
| 1. GHR | → | ↓ | ↑ | | |
| 2. PrlR | | → | ↓ | ↑ | |
| 3. SOCS2 | → | ↓ | ↑ | | |
| 4. TSC2 | → | ↓ | ↑ | | |

Interpretation (example 6, table 4):
1C, 3B: High sensitivity to GH; high GHR low SOCS2 (opposite in non-responding tumours)
2C, 3B, 4B: High sensitivity to Prl; high PrlR low SOCS2, low TSC2 (opposite in non-responding tumours)
1C, 2C, 3B: High sensitivity to both GH and Prl

### Example 7

### Sequences

SEQ ID NO. 1: Human Prolactin Receptor (PrIR)
http://www.uniprot.org/uniprot/P16471
SEQ ID NO. 2: Human Growth Hormone Receptor (GHR)
http://www.uniprot.org/uniprot/P10912
SEQ ID NO. 3: human Suppressor Of Cytokine Signalling 2 (SOCS2)
http://www.uniprot.org/uniprot/O14508
SEQ ID NO. 4: Tuberin/Tuberous sclerosis 2 protein (TCS2)
http://www.uniprot.org/uniprot/P49815
SEQ ID NO. 5: Human Prl including signal peptide (wild-type)
http://www.uniprot.org/uniprot/P01236
MNIKGSPWKGSLLLLLVSNLLLCQSVAPLPICPGGAARCQVTLRDLFDRAVVLSHYIHNL
SSEMFSEFDKRYTHGRGFITKAINSCHTSSLATPEDKEQAQQMNQKDFLSLIVSILRSWN
EPLYHLVTEVRGMQEAPEAILSKAVEIEEQTKRLLEGMELIVSQVHPETKENEIYPVWSG
LPSLQMADEESRLSAYYNLLHCLRRDSHKIDNYLKLLKCRIIHNNNC
SEQ ID NO. 6: Human mature Prl (wild-type)
http://www.uniprot.org/uniprot/P01236
LPICPGGAARCQVTLRDLFDRAVVLSHYIHNLSSEMFSEFDKRYTHGRGFITKAINSCHT
SSLATPEDKEQAQQMNQKDFLSLIVSILRSWNEPLYHLVTEVRGMQEAPEAILSKAVEIE
EQTKRLLEGMELIVSQVHPETKENEIYPVWSGLPSLQMADEESRLSAYYNLLHCLRRDSH
KIDNYLKLLKCRIIHNNNC
SEQ ID NO. 7: Human mature Prl (mutated S33A, Q73L, G129R, K190R)
LPICPGGAARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 8: Human mature Prl (mutated S61A, D68N, Q73L, G129R, K190R)
LPICPGGAARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 9: Human mature Growth Hormone (wild-type)
EFTIPLSRLEDNAMLRAHRLHQLAFETYQEFEEAYIPKEQKYSFLQNPQTSLCFSESIPT
PSNREETQQKSNLELLRISLLLIQSWLEPVQFLRSVFANSLVYGASDSNVYDLLKDLEEG
IQTLMGRLEDGSPRTGQIFKQTYSKETTNSHNDDALLKNYGLLYCFRKEMDKVETFLRIV
QCRSVEGSCGF
SEQ ID NO. 10: Human Growth Hormone (mutated G120R)
EFTIPLSRLEDNAMLRAHRLHQLAFETYQEFEEAYIPKEQKYSFLQNPQTSLCFSESIPT
PSNREETQQKSNLELLRISLLLIQSWLEPVQFLRSVFANSLVYGASESNVYDLLKELEER
IQTLMGRLEDGSPRTGQIFKQTYSKETTNSHNDDALLKNYGLLYCFRKEMDKVETFLRIV
QCRSVEGSCGF
SEQ ID NO. 11: Linker
SGGSGGS
SEQ ID NO. 12: Linker
GGSGGSGGSGGSGGG
SEQ ID NO. 13: Linker
GGSGGSGGSGGSGGSGGS
SEQ ID NO. 14: Linker
GGGGSGGGGSGGGGS
SEQ ID NO. 15: Linker
EFAGAAAV
SEQ ID NO. 16: Human N-terminally truncated Prl (mutated S33A, Q73L,
G129R, K190R)
PICPGGAARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 17: Human N-terminally truncated Prl (mutated S33A, Q73L,
G129R, K190R)
ICPGGAARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 18: Human N-terminally truncated Prl (mutated S33A, Q73L,
G129R, K190R)
CPGGAARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 19: Human N-terminally truncated Prl (mutated S33A, Q73L,
G129R, K190R)
PGGAARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 20: Human N-terminally truncated Prl (mutated S33A, Q73L,
G129R, K190R)
GGAARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 21: Human N-terminally truncated Prl (mutated S33A, Q73L,
G129R, K190R)
GAARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 22: Human N-terminally truncated Prl (mutated S33A, Q73L,
G129R, K190R)
AARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 23: Human N-terminally truncated Prl (mutated S33A, Q73L,
G129R, K190R)
ARCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 24: Human N-terminally truncated Prl (mutated S33A, Q73L,
G129R, K190R)
RCQVTLRDLFDRAVVLSHYIHNLASEMFSEFDKRYTHGRGFITKAINSCHT
SSLATPEDKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 25: Human N-terminally truncated Prl (mutated S61A, D68N,
Q73L, G129R, K190R)
PICPGGAARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 26: Human N-terminally truncated Prl (mutated S61A, D68N,
Q73L, G129R, K190R)
ICPGGAARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 27: Human N-terminally truncated Prl (mutated S61A, D68N,
Q73L, G129R, K190R)
CPGGAARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 28: Human N-terminally truncated Prl (mutated S61A, D68N,
Q73L, G129R, K190R)
PGGAARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 29: Human N-terminally truncated Prl (mutated S61A, D68N,
Q73L, G129R, K190R)
GGAARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 30: Human N-terminally truncated Prl (mutated S61A, D68N,
Q73L, G129R, K190R)
GAARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 31: Human N-terminally truncated Prl (mutated S61A, D68N,
Q73L, G129R, K190R)
AARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 32: Human N-terminally truncated Prl (mutated S61A, D68N,
Q73L, G129R, K190R)
ARCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC
SEQ ID NO. 33: Human N-terminally truncated Prl (mutated S61A, D68N,
Q73L, G129R, K190R)
RCQVTLRDLFDRAVVLSHYIHNLSSEMFSEFDKRYTHGRGFITKAINSCHT
ASLATPENKEQALQMNQKDFLSLIVSILRSWNEPLYHLVTEVRGMQEAPEAILSKAVEIE
EQTKRLLERMELIVSQVHPETKENEIYPVWSGLPSLQMADEESRLSAYYNLLHCLRRDSH
KIDNYLKLLRCRIIHNNNC

### REFERENCES

1. Orlando L, Schiavone P, Fedele P, Calvani N, Nacci A, Rizzo P, Marino A, D'Amico M, Sponziello F, Mazzoni E, Cinefra M, Fazio N, Maiello E, Silvestris N, Colucci G, Cinieri S Molecularly targeted endocrine therapies for breast cancer. Cancer Treat Rev 36 Suppl 3:S67-71
2. Schroder F, Crawford E D, Axcrona K, Payne H, Keane T E Androgen deprivation therapy: past, present and future. BJU Int 109 Suppl 6:1-12
3. Murphy C G, Morris P G Recent advances in novel targeted therapies for HER2-positive breast cancer. Anticancer Drugs 23:765-76
4. Honda K, Ono M, shitashige M, Masuda M, Kamita M, Miura N, Yamada T Proteomic approaches to the discovery of cancer biomarkers for early detection and personalized medicine. Jpn J Clin Oncol 43:103-9
5. Tracy M Latest advances in biomarker discovery and development. Drugs Today (Barc) 48:735-9
6. Friesen H, Hwang P 1973 Human prolactin. Annu Rev Med 24:251-70
7. Goffin V, Binart N, Touraine P, Kelly P A 2002 Prolactin: the new biology of an old hormone. Annu Rev Physiol 64:47-67
8. Binart N, Ormandy C J, Kelly P A 2000 Mammary gland development and the prolactin receptor. Adv Exp Med Biol 480:85-92
9. Walker A M 2005 Prolactin receptor antagonists. Curr Opin Investig Drugs 6:378-85
10. Liu Y, Gong W, Breinholt J, Norskov-Lauritsen L, Zhang J, Ma Q, Chen J, Panina S, Guo W, Li T, Zhang J, Kong M, Liu Z, Mao J, Christensen L, Hu S, Wang L Discovery of the improved antagonistic prolactin variants by library screening. Protein Eng Des Sel 24:855-60
11. Hormone-dependent cancer: Edited by Jorge R. Pasqualini and Benita S. Katzenellenbogen. Published April 1996 by Marcel Dekker Inc. 579 pp. ISBN: 0-8247-9697-7.
12. Fares et al (2010) Endocrinology, 151(9):4410-4417

1. A composition comprising at least one prolactin receptor antagonist and/or at least one growth hormone receptor antagonist, for use in a method of treatment of a prolactin-associated disorder in an individual, the method comprising the steps of:
a) providing a sample of tumour tissue obtained from the individual,
b) determining in said sample, the expression level of the polypeptides growth hormone receptor (GHR), prolactin receptor (PrlR), suppressor of cytokine signalling 2 (SOCS2) and tuberous sclerosis complex 2 (TSC2),
c) comparing the expression levels of step b) with the expression level of a control tissue,
d) assessing a treatment regime by correlating the results of step c) with the corresponding expression pattern of table 1,
e) administering to the individual a therapeutically effective amount of said composition as determined in step d).

2. The composition according to claim 1, wherein the prolactin associated disorder is selected from the group consisting of colon cancer, liver cancer, breast cancer, endometrial cancer, ovary cancer, prostate cancer, parathyroid cancer, benign breast tumour, leiomyoma, renal angiomyolipoma, acromegaly, hyperprolactinemia, obesity resulting from endocrine malfunction, lymphangioleiomyomatosis, lupus erythematosus, benign prostate tumour and peripartum cardiomyopathy.

3. The composition according to any one of the preceding claims, wherein the expression pattern and corresponding treatment regime is:

| | |
|---|---|
| Expression pattern | Treatment |
| No. | GHR | PrlR | SOCS2 | TCS2 | regime |
|---|---|---|---|---|---|
| 3 | ↑ | ↑ | ↑ | ↓ | GHR-A/PrlR-A |
| 4 | ↑ | ↑ | ↓ | ↑ | GHR-A/PrlR-A |
| 5 | ↑ | ↑ | ↓ | ← | GHR-A/PrlR-A |
| 6 | ↑ | ↑ | ↓ | ↓ | GHR-A/PrlR-A |
| 9 | ↑ | ↑ | ← | ↓ | GHR-A/PrlR-A |
| 12 | ↑ | ← | ↑ | ↓ | GHR-A |
| 13 | ↑ | ← | ↓ | ↑ | GHR-A |
| 14 | ↑ | ← | ↓ | ← | GHR-A |
| 15 | ↑ | ← | ↓ | ↓ | GHR-A |
| 18 | ↑ | ← | ← | ↓ | GHR-A |
| 21 | ↑ | ↓ | ↑ | ↓ | GHR-A |
| 22 | ↑ | ↓ | ↓ | ↑ | GHR-A |
| 23 | ↑ | ↓ | ↓ | ← | GHR-A |
| 24 | ↑ | ↓ | ↓ | ↓ | GHR-A |
| 27 | ↑ | ↓ | ← | ↓ | GHR-A |
| 30 | ↓ | ↑ | ↑ | ↓ | PrlR-A |
| 31 | ↓ | ↑ | ↓ | ↑ | PrlR-A |
| 32 | ↓ | ↑ | ↓ | ← | PrlR-A |
| 33 | ↓ | ↑ | ↓ | ↓ | PrlR-A |
| 36 | ↓ | ↑ | ← | ↓ | PrlR-A |
| 39 | ↓ | ← | ↑ | ↓ | PrlR-A |
| 41 | ↓ | ← | ↓ | ← | PrlR-A |
| 42 | ↓ | ← | ↓ | ↓ | PrlR-A |
| 45 | ↓ | ← | ← | ↓ | PrlR-A |
| 57 | ← | ↑ | ↑ | ↓ | PrlR-A |
| 58 | ← | ↑ | ↓ | ↑ | PrlR-A |
| 59 | ← | ↑ | ↓ | ← | PrlR-A |
| 60 | ← | ↑ | ↓ | ↓ | PrlR-A |
| 63 | ← | ↑ | ← | ↓ | PrlR-A |
| 66 | ← | ← | ↑ | ↓ | GHR-A/PrlR-A |
| 67 | ← | ← | ↓ | ↑ | GHR-A/PrlR-A |
| 68 | ← | ← | ↓ | ← | GHR-A/PrlR-A |
| 69 | ← | ← | ↓ | ↓ | GHR-A/PrlR-A |
| 72 | ← | ← | ← | ↓ | GHR-A/PrlR-A |
| 76 | ← | ↓ | ↓ | ↑ | GHR-A |
| 77 | ← | ↓ | ↓ | ← | GHR-A |
| 78 | ← | ↓ | ↓ | ↓ | GHR-A |
| 81 | ← | ↓ | ← | ↓ | GHR-A |

wherein arrow down (↓) indicates decreased expression compared to control tissue, and wherein arrow left (←) indicates essentially unaltered expression compared to control tissue, and wherein arrow up (↑) indicates increased expression compared to control tissue, and wherein GHR-A is a Growth Hormone Receptor Antagonist, and wherein PrlR-A is a Prolactin Receptor Antagonist and wherein GHR-A/PrlR-A is a combination treatment of a Growth Hormone Receptor Antagonist and a Prolactin Receptor Antagonist.

4. The composition according to any of the preceding claims wherein the growth hormone receptor (GHR) has the sequence of SEQ ID NO: 1.

5. The composition according to any of the preceding claims wherein the prolactin receptor (PrlR) has the sequence of SEQ ID NO: 2.

6. The composition according to any of the preceding claims wherein the suppressor of cytokine signalling 2 (SOCS2) has the sequence of SEQ ID NO: 3.

7. The composition according to any of the preceding claims wherein the tuberous sclerosis complex 2 (TSC2) has the sequence of SEQ ID NO: 4.

8. The composition according to any one of claims 1 to 7 wherein the prolactin receptor antagonist is selected from the group consisting of:
a) an isolated polypeptide comprising:
i) an amino acid sequence selected from the group consisting of SEQ ID NOs: 7, 8, 16, 17, 18, 19, 20, 21, 22, 23, 24, 25, 26, 27, 28, 29, 30, 31, 32, 32 and 33; or
ii) a biologically active sequence variant of the amino acid sequence of i) wherein the variant has at least 70% sequence identity to said SEQ ID NO: 7, 8, 16, 17, 18, 19, 20, 21, 22, 23, 24, 25, 26, 27, 28, 29, 30, 31, 32, 32 and 33,
iii) a biologically active fragment of at least 15 contiguous amino acids of any one of i) through ii), said fragment having at least 70% sequence identity to SEQ ID NO: 7 or 8 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

9. The composition according to any one of claims 1 to 7 wherein the growth hormone antagonist of the invention is selected from the group consisting of:
a) an isolated polypeptide comprising:
i) the amino acid sequence of SEQ ID NOs: 10; or
ii) a biologically active sequence variant of the amino acid sequence of i) wherein the variant has at least 70% sequence identity to said SEQ ID NO: 10,
iii) a biologically active fragment of at least 15 contiguous amino acids of any one of i) through ii), said fragment having at least 70% sequence identity to SEQ ID NO: 10 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

10. The composition according to any one of claims 8 and 9, wherein the PrlR-antagonist and/or GHR-antagonist is a polypeptide wherein the polypeptide is a naturally occurring allelic variant of a sequence selected from the group consisting of SEQ ID NOs: 7, 8, 16, 17, 18, 19, 20, 21, 22, 23, 24, 25, 26, 27, 28, 29, 30, 31, 32, 32, 33 and 10.

11. The composition according to any one of claims 8 to 10, wherein the polypeptide is a variant polypeptide described therein, wherein any amino acid specified in the selected sequence is altered to provide a conservative substitution, with the proviso that no more than 50 amino acids are so altered.

12. The composition according to any one of claims 8 to 10, wherein the polypeptide is a variant polypeptide described therein, wherein any amino acid specified in the selected sequence is altered to provide a conservative substitution, with the proviso that no more than 25 amino acids are so altered.

13. The composition according to any one of claims 8 to 12, wherein said polypeptide has at least 65%, more preferably at least 70%, more preferably at least 75%, preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% sequence identity to an amino acid sequence selected from the group consisting of SEQ ID NOs: 7, 8, 16, 17, 18, 19, 20, 21, 22, 23, 24, 25, 26, 27, 28, 29, 30, 31, 32, 32, 33 and 10.

14. The composition according to any one of claims 8 to 13, wherein the composition is a biologically active fragment, wherein the fragment comprises less than 199 contiguous amino acid residues, such as less than 198 contiguous amino acid residues, for example less than 197 contiguous amino acid residues, such as less than 196 contiguous amino acid residues, for example less than 195 contiguous amino acid residues, such as less than 194 contiguous amino acid residues, for example less than 193 contiguous amino acid residues, such as less than 192 contiguous amino acid residues, for example less than 191 contiguous amino acid residues, such as less than 190 contiguous amino acid residues, for example less than 189 contiguous amino acid residues, such as less than 188 contiguous amino acid residues, for example less than 187 contiguous amino acid residues, such as less than 186 contiguous amino acid residues, for example less than 185 contiguous amino acid residues, such as less than 184 contiguous amino acid residues, for example less than 183 contiguous amino acid residues, such as less than 182 contiguous amino acid residues, for example less than 181 contiguous amino acid residues, for example less than 180 contiguous amino acid residues, such as less than 160 contiguous amino acid residues, for example less than 150 contiguous amino acid residues, such as less than 140 contiguous amino acid residues, for example less than 130 contiguous amino acid residues, such as less than 120 contiguous amino acid residues, for example less than 110 contiguous amino acid residues, such as less than 100 contiguous amino acid residues, for example less than 90 contiguous amino acid residues, such as less than 85 contiguous amino acid residues, for example less than 80 contiguous amino acid residues, such as less than 75 contiguous amino acid residues, for example less than 70 contiguous amino acid residues, such as less than 65 contiguous amino acid residues, for example less than 60 contiguous amino acid residues, such as less than 55 contiguous amino acid residues, for example less than 50 contiguous amino acid residues, such as less than 45 contiguous amino acid residues, for example less than 40 contiguous amino acid residues, such as 35 contiguous amino acid residues, for example 30 contiguous amino acid residues, such as 25 contiguous amino acid residues, such as 20 contiguous amino acid residues, for example 15 contiguous amino acid residues of an any one of the amino acid sequences selected from the group consisting of SEQ ID NOs: 7, 8, 16, 17, 18, 19, 20, 21, 22, 23, 24, 25, 26, 27, 28, 29, 30, 31, 32, 32, 33 and 10.

15. The composition according to any one of claims 8 to 13, wherein the polypeptide is a biologically active fragment, wherein the fragment comprises at least 15 contiguous amino acid residues, such as more than 20 contiguous amino acid residues, for example more than 25 contiguous amino acid residues, for example more than 50 contiguous amino acid residues, such as more than 75 contiguous amino acid residues, for example more than 100 contiguous amino acid residues, such as more than 125 contiguous amino acid residues, for example more than 150 contiguous amino acid residues, such as more than 175 contiguous amino acid residues of any one of the amino acid sequences selected from the group consisting of SEQ ID NOs: 7, 8, 16, 17, 18, 19, 20, 21, 22, 23, 24, 25, 26, 27, 28, 29, 30, 31, 32, 32, 33 and 10.

16. The composition according to any one of the preceding claims, wherein amino acid residue in the position corresponding to position 55 of SEQ ID NO. 6 has been substituted with valine, leucine, tryptophan, tyrosine, phenylalanine, proline or threonine.

17. The composition according to any one of the preceding claims, wherein amino acid residue in the position corresponding to position 56 of SEQ ID NO. 6 has been substituted with glycine or a glutamine.

18. The composition according to any one of the preceding claims, wherein amino acid residue in the position corresponding to position 57 of SEQ ID NO. 6 has been substituted with valine, leucine, isoleucine, tryptophan, tyrosine, phenylalanine, proline or threonine.

19. The composition according to any one of the preceding claims, wherein one or more of the amino acids in the positions corresponding to positions 61, 71 and 73 of SEQ ID NO. 6 have been altered to a different amino acid.

20. The composition according to any one of claims 8 to 19 wherein the polypeptide is glycosylated.

21. The composition according to any one of claims 8 to 20, wherein the polypeptide is capable of forming at least one intramolecular cystin bridge.

22. The composition according any one of claims 8 to 20, comprising a dimer of said polypeptide linked through at least one intermolecular cystin bridge.

23. The composition according to any one of claims 8 to 22, wherein said polypeptide further comprises an affinity tag, such as a polyhis tag, a GST tag, a HA tag, a Flag tag, a C-myc tag, a HSV tag, a V5 tag, a maltose binding protein tag, a cellulose binding domain tag.

24. The composition according to any one of claims 1 to 23, wherein the polypeptide is chemically modified in order to increase its half-life when administered to a patient, in particular its plasma half-life.

25. The composition according to any one of claims 1 to 23, wherein said polypeptide further comprises a moiety conjugated to said polypeptide, thus generating a moiety-conjugated polypeptide.

26. The composition according to claim 25, wherein the moiety-conjugated polypeptide has a plasma and/or serum half-life being longer than the plasma and/or serum half-life of the non-moiety conjugated polypeptide.

27. The composition according to claim 26, wherein the moiety facilitates transport across the blood brain barrier.

28. The composition according to claim 27, wherein the moiety is an antibody from a camelid species such as a recombinant or native single-chain antibody from dromedaries, camels, llamas, alpacas, vicuñas, or guanacos.

29. The composition according to any one of claims 25 to 28, wherein the moiety conjugated to the composition is one or more type of moieties selected from the group consisting of albumin, fatty acids, polyethylene glycol (PEG), acylation groups, antibodies and antibody fragments.

30. The composition according to any one of claims 25 to 29, wherein the polypeptide and the moiety are conjugated to each-other by a linker.

31. The composition according to any one of claims 25 to 30, wherein the moiety is conjugated to the prolactin receptor antagonist and/or the growth hormone receptor antagonist.

32. The composition according to any one of claim 8 or 9, wherein the vector further comprises a promoter operably linked to the nucleic acid sequence.

33. The composition according to claim 32, wherein the promoter is selected from the group consisting of: CMV, human UbiC, RSV, Tet-regulatable promoter, Mo-MLV-LTR, Mx1, EF-1alpha, PDGF beta and CaMK II.

34. The composition according to any one of claim 8 or 9, wherein the vector is selected from the group consisting of vectors derived from the Retroviridae family including lentivirus, HIV, SIV, FIV, EAIV, CIV.

35. The composition according to any one of claim 8 or 9, wherein the vector is selected from the group consisting of adeno associated virus, adenovirus, alphavirus, baculovirus, HSV, coronavirus, Bovine papilloma virus, Mo-MLV.

36. The composition according to any one of claim 8 or 9, wherein the host cell is selected from the group consisting of Saccharomyces cerevisiae, E. coli, Aspergillus and insect cells such as Sf9 insect cells.

37. The composition according to any one of claim 8 or 9, wherein the host cell is selected from the group consisting of mammalian cells selected from the group consisting of human, feline, porcine, simian, canine, murine and rat cells.

38. The composition according to any one of claim 8 or 9, wherein said host cell is selected from the group consisting of CHO, CHO-K1, HEI193T, HEK293, COS, HiB5, RN33b and BHK cells.

39. The composition according to any one of the preceding claims, wherein the growth hormone receptor antagonist (GHR-A) is a stable sequence variant of SEQ ID NO. 9, wherein said sequence variant comprises at least one amino acid residue which has been altered to a different amino acid residue.

40. The composition according to claim 39 wherein the sequence variant is less than 99% identical to SEQ ID NO. 6, preferably less than 98% identical to SEQ ID NO. 6, preferably less than 97% identical to SEQ ID NO. 6, preferably less than 96% identical to SEQ ID NO. 6, preferably less than 95% identical to SEQ ID NO. 6, preferably less than 94% identical to SEQ ID NO. 6, preferably less than 93% identical to SEQ ID NO. 6, preferably less than 92% identical to SEQ ID NO. 6, preferably less than 91% identical to SEQ ID NO. 6, preferably less than 90% identical to SEQ ID NO. 6, preferably less than 85% identical to SEQ ID NO. 6, preferably less than 80% identical to SEQ ID NO. 6, preferably less than 75% identical to SEQ ID NO. 6, preferably less than 70% identical to SEQ ID NO. 6, with the proviso that the sequence variant of SEQ ID NO. 6 is capable of binding to and inhibiting cellular signaling through the prolactin receptor.

41. The composition according to any one of the preceding claims, wherein the prolactin receptor antagonist (PrlR-A) is a stable sequence variant of SEQ ID NO. 6 wherein said sequence variant comprises at least one amino acid residue which has been altered to a different amino acid residue.

42. The composition according to claim 41 wherein the sequence variant is less than 99% identical to SEQ ID NO. 5, preferably less than 98% identical to SEQ ID NO. 5, preferably less than 97% identical to SEQ ID NO. 5, preferably less than 96% identical to SEQ ID NO. 5, preferably less than 95% identical to SEQ ID NO. 5, preferably less than 94% identical to SEQ ID NO. 5, preferably less than 93% identical to SEQ ID NO. 5, preferably less than 92% identical to SEQ ID NO. 5, preferably less than 91% identical to SEQ ID NO. 5, preferably less than 90% identical to SEQ ID NO. 5, preferably less than 85% identical to SEQ ID NO. 5, preferably less than 80% identical to SEQ ID NO. 5, preferably less than 75% identical to SEQ ID NO. 5, preferably less than 70% identical to SEQ ID NO. 5, with the proviso that the sequence variant of SEQ ID NO:5 is capable of binding to and inhibiting cellular signaling through the prolactin receptor.

43. The composition according to claim 1, wherein the prolactin receptor antagonist is an anti-prolactin receptor antibody or an antibody fragment thereof.

44. The composition according to claim 1, wherein the growth hormone receptor antagonist is an anti-growth hormone receptor antibody or an antibody fragment thereof.

45. The composition according to any one of claims 43 to 44, wherein the antibody is a monoclonal antibody.

46. The composition according to any one of claims 43 to 44, wherein the antibody is a polyclonal antibody.

47. The composition according to any one of the preceding claims further comprising an additional active ingredient.

48. The composition according to claim 47 wherein the additional active ingredient is selected from the group consisting of a dopamine agonist, rapamycin or a derivative of any one of the dopamine agonist or rapamycin, wherein the derivative is capable of acting on the mTOR pathway, a GH receptor antagonist or an anti-cancer drug.

49. The composition according to any one of the preceding claims wherein said composition is a pharmaceutical composition.

50. The composition according to claim 49 further comprising a pharmaceutically acceptable carrier.

51. The composition according to claim 49 further comprising a pharmaceutically acceptable vehicle.

52. The composition according to any one of claims 49 and 52 wherein the pH of the composition is between pH 4 and pH 10.

53. The composition according to any one of claims 49 to 52 wherein the composition is formulated for oral administration.

54. The composition according to any one of claims 49 to 52 wherein the composition is formulated for parenteral administration.

55. The composition according to claim 54 wherein the parenteral administration is by injection.

56. The composition according to any one of claims 54 and 55, wherein the administration is intravenous, intramuscular, intraspinal, intraperitoneal, subcutaneous, a bolus or a continuous administration.

57. The composition according to any one of claims 49 to 56, wherein the administration occurs at intervals of 30 minutes to 24 hours, such as at intervals of 1 to 6 hours, such as three times a day.

58. The composition according to any one of claims 49 to 57, wherein the duration of the treatment is from 6 to 72 hours.

59. The composition according to any one of claims 49 to 57, wherein the duration of the treatment is from 24 hours to 7 days.

60. The composition according to any one of claims 49 to 57, wherein the duration of the treatment is from 4 days to 150 days.

61. The composition according to any one of claims 49 to 57, wherein the duration of the treatment is lifelong.

62. The composition according to any one of claims 49 to 61, wherein the dosage of the active ingredient is between 10 μg to 500 mg per kg body mass, such as from 50 μg to 250 mg per kg body mass.

63. A kit comprising the composition according to any one of claims 49 to 62, and instructions for use.

64. The kit according to claim 63, wherein the instructions comprises table 1 or a subset of table 1.

65. A method of treatment of a prolactin-associated disorder in an individual in need thereof, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) assessing a treatment regime, administering to the individual a therapeutically effective amount of a prolactin receptor antagonist and/or a growth hormone receptor antagonist.

66. A method for selecting treatment of a prolactin-associated disorder in an individual, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) selecting a treatment regime based on table 1.

67. A method for diagnosing a prolactin-associated disorder in an individual, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue, wherein an expression pattern equal to the expression patterns 3, 4, 5, 6, 9, 12, 13, 14, 15, 18, 21, 22, 23, 24, 27, 30, 31, 32, 33, 36, 39, 41, 42, 45, 57, 58, 59, 60, 63, 66, 67, 68, 69, 72, 76, 77, 78 or 81 of table 1 is indicative of a prolactin associated disorder.

68. A computer implemented method for selecting treatment of a prolactin-associated disorder, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue,
d) selecting a treatment regime based on table 1.

69. A computer implemented method for diagnosing a prolactin-associated disorder, the method comprising the steps of:
a) providing a sample from tumour tissue of an individual,
b) determining in the sample of step a), the expression level of the polypeptides growth hormone receptor (GHR), prolactin receptor (PrlR), suppressor of cytokine signalling 2 (SOCS2) and tuberous sclerosis complex 2 (TSC2),
c) correlating the expression levels of step b) with the expression level of a control tissue, wherein an expression pattern equal to the expression patterns 3, 4, 5, 6, 9, 12, 13, 14, 15, 18, 21, 22, 23, 24, 27, 30, 31, 32, 33, 36, 39, 41, 42, 45, 57, 58, 59, 60, 63, 66, 67, 68, 69, 72, 76, 77, 78 or 81 of table 1 is indicative of a prolactin associated disorder.

70. The computer implemented method of any one of claims 68 and 69, further comprising the features of any one of claims 2-48

71. A computer program product having a computer readable medium, said computer program product suitable for selecting a treatment of a prolactin associated disorder in a subject based on expression patterns of the polypeptides growth hormone receptor (GHR), prolactin receptor (PrlR), suppressor of cytokine signalling 2 (SOCS2) and tuberous sclerosis complex 2 (TSC2) in tumour tissue in a sample obtained from said subject, said computer program product comprising means for carrying out all the steps of the method as defined in any of claims 66 to 70.
